(19) United States
(12) Patent Application Publication
(10) Pub. No.: US 2006/0252786 A1
(43) Pub. Date: Nov. 9, 2006

(54) Nicotinic-opioid synergy for analgesia

(75) Inventors: Pamela Flood, Closter, NJ (US); Steven Shafer, Palo Alto, CA (US)

Correspondence Address: COOPER & DUNHAM, LLP, 1185 AVENUE OF THE AMERICAS, NEW YORK, NY, 10036, US

(73) Assignee: The Trustees of Columbia University, New York, NY (US)

(21) Appl. No.: 11/255,002

(22) Filed: Oct. 20, 2005

Related U.S. Application Data
(60) Provisional application No. 60/620,836, filed on Oct. 21, 2004.

(51) Int. Cl. A61K 31/485; A61K 31/4439; A61K 31/445; A61K 31/22; A61K 31/14

(52) U.S. Cl. 514/282; 514/343; 514/546; 514/642; 514/317

(57) ABSTRACT

This invention provides two methods for reducing, or inhibiting the onset of, pain in a subject. The first method for reducing, or inhibiting the onset of, pain in a subject comprises administering to the subject (a) a nicotinic receptor agonist, and (b) an opioid receptor agonist; wherein the ratio of nicotinic receptor agonist to opioid receptor agonist administered to the subject is less than 3:4 and greater than 1:100. The second method for reducing, or inhibiting the onset of, pain in a subject comprises administering to the subject (a) a nicotinic receptor agonist at a rate of less than 3 mg per three hour period; and (b) an opioid receptor agonist at a rate of less than 4 mg per three hour period. This invention also provides two compositions, a transdermal patch, and an article of manufacture for practicing the instant methods.

This invention claims the benefit of U.S. Provisional Application No. 60/620,836, filed Oct. 21, 2004, the contents of which are hereby incorporated by reference into this application.

Throughout this application, various publications are referenced. Full bibliographic citations for these publications are found at the end of the specification immediately preceding the claims. The disclosures of these publications in their entireties are hereby incorporated by reference into this application in order to more fully describe the state of the art known to those skilled therein as of the date of the invention described and claimed herein.

### BACKGROUND OF THE INVENTION

Pain is a major public health issue affecting 1 in 5 Americans. Seventy to eighty percent of the 23 million Americans who undergo surgical procedures each year experience moderate to severe pain despite treatment with all of the analgesic medications that are currently available (Owen, McMillan et al. 1990; Thomas, Robinson et al. 1998; Svensson, Sjostrom et al. 2000). “In the past, postoperative pain has been managed badly and scandal avoided only because patients did not expect better and it is by definition short-lived.” (Twycross 1999). Extended hospitalization, compromised prognosis, higher morbidity and mortality and the development of a chronic pain state as a result of altered neuronal plasticity are negative clinical outcomes of inadequately managed acute postoperative pain (Stephens, Laskin et al. 2003). In recognition of this crisis, the decade from 2001 to 2010 has been designated The Decade of Pain Control and Research, by congressional mandate.

Opioid agonists are commonly used and highly efficacious for pain treatment after surgery; however doses are limited by side effects. The most serious potential side effect of opioid agonists is respiratory depression. When used for acute pain, opioids reduce minute volume due to a reduction in both tidal volume and respiratory rate. This is particularly problematic when opioids are used acutely in the postoperative patient because of postoperative atelectasis and residual effects of surgery and anesthetic drugs (Longnecker, Grazis et al. 1973; Lindberg, Gunnarsson et al. 1992). Additionally, a common clinical observation in the immediate post-operative period is that patients will complain of severe pain when awake, but will then have severe airway obstruction when they fall asleep. This results from increased sensitivity to opioid induced ventilatory depression when patients are asleep (Forrest and Bellville 1964). Post-operative patients are already at risk of disordered breathing and arterial desaturation and opioid-induced ventilatory depression and sedation can only exacerbate this (Bowdle 2004).

Other acute opioid side effects include nausea, pruritis, sedation, and ileus. Patients and physicians may choose inadequate pain control to minimize these unpleasant side effects. This is demonstrated by the placebo group in our study where patients with the ability to control their morphine dosing with patient controlled analgesia do not dose themselves to optimal pain relief because of these considerations (Flood and Daniel 2004).

The use of analgesic adjuvants to provide synergy is only logical if there is synergy for the desired therapeutic effect, and not for the toxic effects. While opioids generally depress central nervous system (CNS) activity, nicotine is a stimulant (Haxhiu, Mitra et al. 1985) which would be expected to partly reverse opioid-induced ventilatory depression and sedation. The only area of overlapping toxicity between opioids and nicotine is nausea, which is why our study specifically looked for increased nausea in patients receiving nicotine vs. control subjects.

Combinations of drugs with similar positive effects have been commonly used in clinical pharmacology to reduce the dose and thus side effects that occur when each drug is used alone. Although no systemic alternative to opioids for the treatment of postoperative pain has been discovered, analgesic adjuvants have been successfully used in the postoperative period to reduce opioid consumption and side effects (O'Hara, Fanciullo et al. 1997; Alexander, El-Moalem et al. 2002; Ng, Parker et al. 2002). The most commonly used adjuvants are NSAIDs. Although NSAIDs lack sufficient analgesic efficacy to be effective in the immediate post-operative period as sole agents, they are useful adjuvants to opioid analgesics. The doses of traditional non-COX selective non-steroidal anti-inflammatory medications are limited by side effects such as gastritis, decreased kidney blood flow, impaired bone healing, and impaired platelet function (Saray, Buyukkocak et al. 2001; Foral, Wilson et al. 2002). It may be that the COX-2 selective NSAIDs have reduced side effects in the post-operative setting.

Although nicotine has been known for years to possess analgesic efficacy in both animal studies (Rao, Correa et al. 1996; Hunt, Wu et al. 1998; Li, Rainnie et al. 1998) and human volunteer pain studies (Pomerleau, Turk et al. 1984; Fertig, Pomerleau et al. 1986; Perkins, Grobe et al. 1994; Jamner, Girdler et al. 1998), prior to our preliminary animal and human studies nicotine had never been assessed as an analgesic adjuvant to opioids.

Mechanism of Nicotinic Analgesia

The antinociceptive action of nicotine is thought to be due to nicotinic activation in the central nervous system. Nicotinic agonists that cross the blood brain barrier can cause antinociception through actions in both the brain and spinal cord. In contrast, hexamethonium, a nicotinic antagonist that does not cross the blood brain barrier, has no effect on the antinociceptive action of nicotine (Bitner, Nikkel et al. 1998).

Nicotinic agonists applied in the brain can have either pro- or antinociceptive effects (Parvini, Hamann et al. 1993; Khan, Taylor et al. 1994; Khan, Marsala et al. 1996). Administered into the mid-fourth ventricle, nicotine produces analgesia in low doses and hyperalgesia in higher doses (Parvini, Hamann et al. 1993; Rao, Correa et al. 1996). Activation of the pedunculopontine tegmental nucleus and the nucleus raphe magnus with nicotine causes analgesia that is inhibited by the administration of antagonists of α₂-adrenergic, serotonergic and muscarinic receptors to the spinal cord (Iwamoto 1991; Iwamoto and Marion 1993). Intracerebroventricular injection of nicotine causes increases in the release of spinal serotonin, when measured with in vivo microdialysis (Rueter, Meyer et al. 2000).

Similarly, intrathecal injection of nicotinic agonists can cause both pro- and antinociceptive effects. When rats were treated with nicotine systemically, intracerebraventricularly and intrathecally, the intrathecal route was the most potent in causing analgesia (Aceto, Bagley et al. 1986). Intrathecal nicotine causes antinociception in rats that was reduced by the α₂-adrenergic inhibitor yohimbine, suggesting nicotinic facilitation of norepinephrine release that stimulates postsynaptic α₂-adrenergic receptors (Christensen and Smith 1990). In the lumbar spinal cord, slice experiments have suggested that the release of serotonin is also controlled by tonically active nicotinic receptors (Cordero-Erausquin and Changeux 2001).

Nicotinic receptors are expressed on multiple axonal terminals in the CNS, where they facilitate the release of glutamate, acetylcholine, norepinephrine, serotonin, GABA and glycine (see MacDermott, 1999 for review) (MacDermott, Role et al. 1999). In the brain nicotinic receptors are expressed in cellulodendritic domains as well as terminal domains of adrenergic neurons in the locus ceruleus, areas A5 and A7, serotonergic neurons in the nucleus raphe magnus and in cholinergic neurons (Aceto, Bagley et al. 1986; Iwamoto and Marion 1993; Mitchell 1993; Li, Rainnie et al. 1998). In the spinal cord nicotinic binding sites are found predominantly in laminae II and III of the dorsal horn and are almost entirely contained in the thoracic and lumbar areas (Aceto, Bagley et al. 1986; Gillberg, Hartvig et al. 1990).

Thus nicotine activates adrenergic or serotonergic systems either through cellular action in the brain or by increasing transmitter release by acting at the axonal terminals in the spinal cord. Norepinephrine and serotonin have largely inhibitory actions at dorsal horn neurons (Garraway and Hochman 2001). Similarly, nicotine can facilitate the release of acetylcholine that can have either an inhibitory or excitatory effect on dorsal horn cells through actions on muscarinic receptors (Garraway and Hochman 2001) (FIG. 1). The pronociceptive effect of intrathecal nicotine though is thought to be largely due to the facilitation of glutamate release by nicotine and the activation of postsynaptic NMDA receptors (Khan, Taylor et al. 1994; Khan, Marsala et al. 1996; Khan, Buerkle et al. 1998).

Despite the potentially complicated biphasic effects of the descending modulators, norepinephrine, serotonin and acetylcholine have a net inhibitory effect on transmission at the dorsal horn of the spinal cord (Li and Zhuo 2001) that is facilitated by the presynaptic nicotinic activation (Cordero-Erausquin and Changeux 2001; Li and Eisenach 2002). Nicotine and opioid agonists interact with many common pathways that impact on pain sensation. The interaction is also brought out by the clinical observation that deprived smokers have higher postoperative narcotic requirements than non-smokers (Woodside 2000; Creekmore, Lugo et al. 2004).

Nicotinic Acetylcholine Receptors (nAChRs)

Nicotinic acetylcholine receptors are expressed throughout the brain and spinal cord, as well as in autonomic and peripheral neurons where they both mediate synaptic transmission and act pre-synaptically to control the release of other neurotransmitters (Woolf, 1991; McGehee, 1995a; and MacDermott, 1999). Biochemical and pharmacological studies have demonstrated that there are multiple functional subtypes of nicotinic receptors present in the human brain. Nicotinic acetylcholine receptors are composed of a combination of α and β subunits arranged in a pentameric ring. Generally the receptor is composed of three α and two α subunits. Currently nine different α subunit types and 3 different β subunit types have been identified in the brain and ganglia tissue. Selected examples of nAChRs comprised of α and β subunit combinations are listed in Table 1.

TABLE 1
Examples of Nicotinic Receptor Types

| | | | | | | | | |
|---|---|---|---|---|---|---|---|---|
| α₂β₂ | α₃β₂ | α₄β₂ | α₅β₂ | α₆β₂ | α₇β₂ | α₈β₂ | α₉β₂ | α₁₀β₂ |
| α₂β₃ | α₃β₃ | α₄β₃ | α₅β₃ | α₆β₃ | α₇β₃ | α₈β₃ | α₉β₃ | α₁₀β₃ |
| α₂β₄ | α₃β₄ | α₄β₄ | α₅β₄ | α₆β₄ | α₇β₄ | α₈β₄ | α₉β₄ | α₁₀β₄ |

Subunits α₇₋₁₀ can also form homopentameric nicotinic receptors. The receptor forms listed above are merely examples of the potential combinations of α and β subunits that can form nAChRs.

Analgesic Action and Potential of Subtype Selective Nicotinic Agonists

The subunit composition of the nicotinic receptors responsible for the antinociceptive effects of nicotinic agonists is controversial. Studies of mice lacking α₄ and or β₂ nicotinic subunits and some pharmacological studies suggest that receptors containing both subunits are required for the antinociceptive effects of nicotine (Damaj, Fei-Yin et al. 1998; Marubio, del Mar Arroyo-Jimenez et al. 1999). Recent studies show that β₂-containing nicotinic receptors are responsible for the enhancement of the release of norepinephrine in the spinal cord by the nicotinic agonist metanicotine (Li and Eisenach 2002). Nicotinic antagonists selective for α₇ containing nicotinic receptors can also be antinociceptive in some settings (Damaj, Meyer et al. 2000). However some studies with nicotinic antagonists suggest that a nicotinic receptor not composed of α₄β₂ or α₇ subunits is responsible for nicotinic antinociception (Rueter, Meyer et al. 2000). It follows from these disparate reports that several types of nicotinic receptors composed of different subunits play roles in antinociception when activated in different anatomical locations (i.e. spinal vs. ICV nicotine) and under different pain conditions (i.e. tail flick, hind paw withdrawal, pressure sensitivity testing) (Caggiula, Epstein et al. 1995).

Nicotinic Agonists

Nicotine is the prototypical nAChR agonist. A number of receptor-selective nAChR agonists have been isolated, including, but not limited to, DMPP, DMAC, epibatidine (U.S. Pat. No. 6,077,846), and ABT 418 (Americ, 1994). Nicotine and nicotinic agonists have been used to treat various conditions including movement disorders, dysfunction of the central or autonomic nervous systems, neurodegenerative disorders, cardiovascular disorders, convulsive disorders, drug abuse and eating disorders.

Nicotine is commonly used on an outpatient basis for smoking cessation and in children with Tourette's. Nicotine can be administered via an intranasal route. Intranasal nicotine has its peak effect in five minutes and is dissipated in about one hour. As nicotine acts as an agonist at sympathetic ganglia, it can cause increases in heart rate and blood pressure. At a dose of 3 mg intranasally, an average increase of 7 mM of mercury in systolic blood pressure and no change in diastolic blood pressure or heart rate is observed in non-smoking volunteers (Fishbein, 2000). This level of nicotine administration has minimal hemodynamic effects and results in an arterial peak concentration of 100 μM and a steady state venous concentration of 30 μM of nicotine (Guthrie, 1999). As nicotine crosses the blood-brain-barrier, these concentrations would be expected to result in significant activation of nicotinic receptors in the brain and spinal cord.

Human Volunteer Studies of Nicotinic Analgesia

Studies in human volunteers have shown nicotinic analgesia. Nicotine has analgesic effects in experimental paradigms of thermal (Pomerleau, Turk et al. 1984; Fertig, Pomerleau et al. 1986; Pomerleau 1986; Perkins, Grobe et al. 1994) and electrically evoked pain (Jamner, Girdler et al. 1998) in both smokers and non-smokers. The analgesic efficacy of nicotine in these studies was modest and variable. Some studies have suggested that nicotine analgesia is only present in men (Jamner, Girdler et al. 1998) while others have shown efficacy in both men and women (Kanarek and Carrington 2004).

### SUMMARY OF THE INVENTION

This invention provides two methods for reducing, or inhibiting the onset of, pain in a subject. The first method for reducing, or inhibiting the onset of, pain in a subject comprises administering to the subject (a) a nicotinic receptor agonist, and (b) an opioid receptor agonist; wherein the ratio of nicotinic receptor agonist to opioid receptor agonist administered to the subject is less than 3:4 and greater than 1:100, so as to thereby reduce, or inhibit the onset of, pain in the subject.

The second method for reducing, or inhibiting the onset of, pain in a subject comprises administering to the subject (a) a nicotinic receptor agonist at a rate of less than 3 mg per three hour period; and (b) an opioid receptor agonist at a rate of less than 4 mg per three hour period, so as to thereby reduce, or inhibit the onset of, pain in the subject.

This invention provides a composition comprising a pharmaceutically acceptable carrier, a nicotinic receptor agonist and an opioid receptor agonist, wherein the nicotinic receptor agonist and the opioid receptor agonist are present in a ratio of between greater than 1:100 and less than 3:4.

This invention provides a transdermal patch comprising a nicotinic receptor agonist and an opioid receptor agonist, whereby the nicotinic receptor agonist and opioid receptor agonist are released into a subject upon placing the patch on the subject's skin.

This invention provides an article of manufacture for the intravenous administration of a composition to a subject comprising a packaging material having therein a composition comprising (a) a pharmaceutically acceptable carrier suitable for intravenous administration, (b) a nicotinic receptor agonist and (c) an opioid receptor agonist, wherein the nicotinic receptor agonist and the opioid receptor agonist are present in a ratio of 1:8.

This invention provides a second composition comprising a nicotinic receptor agonist and an opioid receptor agonist.

### BRIEF DESCRIPTION OF THE FIGURES

FIG. 1

This figure shows a schematic showing that descending inhibitory input to the spinal cord is modulated by nicotinic acetylcholine receptors.

FIG. 2

This figure shows the postoperative VAS scores in patients receiving nicotine or placebo (all 20 patients). The VAS trajectories for patients receiving nicotine are shown in solid lines, while those for patients receiving placebo are shown in dotted lines. Mean curves for each group are shown in bold lines.

FIG. 3

This figure shows postoperative morphine administration in patients receiving nicotine (solid lines) or placebo (dotted lines).

FIGS. 4A & 4B

These figures show hemodynamic response to nicotine treatment during the first hour after surgery. Patients who received nicotine had lower systolic blood pressure. There was no difference in diastolic blood pressure or heart rate. Legend: ∇=placebo systolic; Δ=placebo diastolic; ▾=nicotine systolic; ▴=nicotine diastolic; ●=nicotine; ◯=placebo.

FIG. 5

This figure shows a response surface for nicotine-morphine von Frey response in the mouse incisional pain model.

FIG. 6

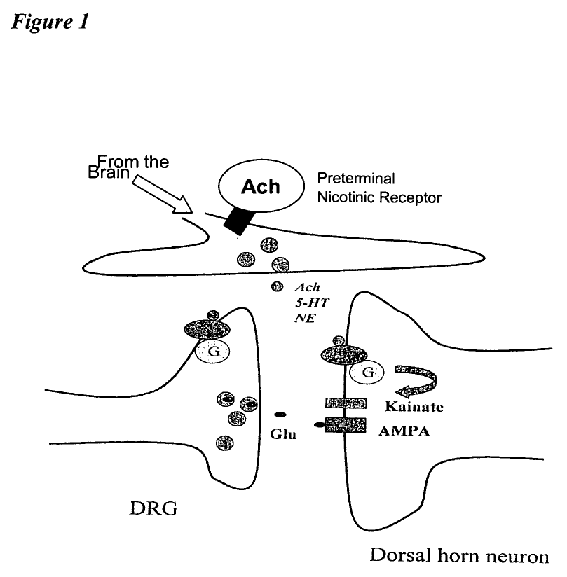

This figure shows tolerance to morphine is reduced by a single treatment with nicotine. Morphine analgesia is reduced to 60% in our post-operative mouse model after 18 hours of continuous infusion. Tolerance is reduced in animals who are pretreated with a single dose of nicotine (P<0.001). Legend: ·=placebo; ▪=placebo exponential fit; □=nicotine 1.5 mg/kg; □=nicotine exponential fit.

### DETAILED DESCRIPTION OF THE INVENTION

Definitions

As used in this application, except as otherwise expressly provided herein, each of the following terms shall have the meaning set forth below.

As used herein, an “agent” shall mean any chemical entity, including, without limitation, a protein, an antibody, a lectin, a nucleic acid, a small molecule, a chemical compound and any combination thereof.

As used herein, an “agonist” is an agent that interacts with a specific cellular receptor, and activates it, i.e., elicits a biochemical response from the receptor (e.g. cleavage of a molecule or phosphorylation of a molecule). An agonist can be an agent endogenous to a given subject (e.g., a hormone) or it can be exogenous (e.g., a synthetic drug).

As used herein, “nicotinic receptor agonist” is an agent that interacts with and activates a nicotinic receptor. A nicotinic receptor agonist can be, for example, nicotine or a derivative thereof. A nicotinic receptor can comprise, for example, any permutation of α and β subunits as set forth above in Table 1, as well as any heterologous variant thereof (e.g. α₂α₃β₂β₃β₄).

As used herein, “opioid receptor agonist” is an agent that interacts with and activates an opioid receptor. An opioid receptor agonist can be, for example, morphine or a derivative thereof.

As used herein, “ratio”, when used in connection with two agonists, means weight ratio. For example, a composition having 1 mg of nicotine and 3 mg of morphine has a nicotine:morphine ratio of 1:3.

As used herein, the term “subject” shall mean any animal including, without limitation, a human, a mouse, a rat, a rabbit, a non-human primate, or any other mammal. In the preferred embodiment, the subject is human. The subject can be male or female.

### EMBODIMENTS OF THE INVENTION

This invention provides two methods for reducing, or inhibiting the onset of, pain in a subject. The first method for reducing, or inhibiting the onset of, pain in a subject comprises administering to the subject (a) a nicotinic receptor agonist, and (b) an opioid receptor agonist; wherein the ratio of nicotinic receptor agonist to opioid receptor agonist administered to the subject is less than 3:4 and greater than 1:100, so as to thereby reduce, or inhibit the onset of, pain in the subject.

The second method for reducing, or inhibiting the onset of, pain in a subject comprises administering to the subject (a) a nicotinic receptor agonist at a rate of less than 3 mg per three hour period; and (b) an opioid receptor agonist at a rate of less than 4 mg per three hour period, so as to thereby reduce, or inhibit the onset of, pain in the subject.

In one embodiment of the first method, the nicotinic receptor agonist and the opioid receptor agonist are administered simultaneously. In another embodiment, the nicotinic receptor agonist is administered before the opioid receptor agonist. In a further embodiment, the opioid receptor agonist is administered before the nicotinic receptor agonist.

In one embodiment of the first method, the amount of nicotinic receptor agonist administered is from about 0.1 mg-1.0 mg per three hour period. In another embodiment, the amount of nicotinic receptor agonist administered is about 0.5 mg per three hour period. In a further embodiment, the amount of nicotinic receptor agonist administered is about 0.25 mg per three hour period.

In one embodiment of the first method, the amount of opioid receptor agonist administered is from about 1.0-8.0 mg per three hour period. In another embodiment, the amount of opioid receptor agonist administered is about 4.0 mg per three hour period.

In one embodiment of the first method, the ratio of nicotinic receptor agonist to opioid receptor agonist administered to the subject is between 1:10 and 1:3. In another embodiment, the ratio of nicotinic receptor agonist to opioid receptor agonist administered to the subject is about 1:4. In a further embodiment, the ratio of nicotinic receptor agonist to opioid receptor agonist administered to the subject is about 1:8. In one embodiment the effective amount of the nicotinic receptor agonist is from about 0.01 mg to less than 3 mg. In another embodiment, the effective amount is from about 0.01 mg to about 2 mg. In another embodiment, the effective amount is from about 0.01 mg to about 1 mg. In another embodiment, the effective amount is from about 0.01 mg to about 0.5 mg. In another embodiment, the effective amount is from about 0.01 mg to about 0.1 mg. In another embodiment, the effective amount is from about 0.1 mg to about 0.5 mg. In another embodiment, the effective amount is from about 0.1 mg to about 1.0 mg. In another embodiment, the effective amount is from about 0.1 mg to about 2.0 mg. In another embodiment, the effective amount is from about 0.1 mg to less than 3.0 mg. In another embodiment, the effective amount is from about 0.5 mg to about 1.0 mg. In another embodiment, the effective amount is from about 0.5 mg to about 2.0 mg. In a further embodiment, the effective amount is from about 0.5 mg to less than 3.0 mg. For each of the above embodiments, an opioid receptor agonist amount is envisioned for which the nicotinic receptor agonist:opioid receptor agonist ratio is between 1:10 and 1:3, and preferably about 1:4 or 1:8.

In one embodiment the effective amount of the opioid receptor agonist is from about 0.01 mg to less than 4 mg. In another embodiment, the effective amount is from about 0.01 mg to about 3 mg. In another embodiment, the effective amount is from about 0.01 mg to about 2 mg. In another embodiment, the effective amount is from about 0.01 mg to about 1 mg. In another embodiment, the effective amount is from about 0.01 mg to about 0.5 mg. In another embodiment, the effective amount is from about 0.01 mg to about 0.1 mg. In another embodiment, the effective amount is from about 0.1 mg to about 0.5 mg. In another embodiment, the effective amount is from about 0.1 mg to about 1.0 mg. In another embodiment, the effective amount is from about 0.1 mg to about 2.0 mg. In another embodiment, the effective amount is from about 0.1 mg to about 3.0 mg. In another embodiment, the effective amount is from about 0.1 mg to less than 4.0 mg. In another embodiment, the effective amount is from about 0.5 mg to about 1.0 mg. In another embodiment, the effective amount is from about 0.5 mg to about 2.0 mg. In another embodiment, the effective amount is from about 0.5 mg to about 3.0 mg. In a further embodiment, the effective amount is from about 0.5 mg to less than 4.0 mg. For each of the above embodiments, a nicotinic receptor agonist amount is envisioned for which the nicotinic receptor agonist:opioid receptor agonist ratio is between 1:10 and 1:3, and preferably about 1:4 or 1:8.

Determining an effective amount of nicotinic receptor agonist and opioid receptor agonist for use in the instant invention can be done based on animal data using routine computational methods. A person of ordinary skill in the art, based on the instant disclosure, can perform simple titration experiments to determine what amounts of agents, in addition to those disclosed herein, would be effective in treating, or inhibiting the onset of, pain.

The amount of the agonist will vary depending on the subject and upon the particular route of administration used. Based upon the agonist, the amount can be delivered continuously, such as by continuous intravenous pump, or at periodic intervals (for example, on one or more separate occasions). Desired time intervals of multiple amounts of a particular agonist can be determined without undue experimentation by one skilled in the art based upon numerous examples herein.

In one embodiment, the agonists are administered per three hour period. Optionally, the above amounts are the amounts to be administered during a three hour period. In another embodiment, the agonists are administered once a day. In a further embodiment, each possible pairing of each of the above-recited nicotinic receptor agonist amounts with each of the above-recited opioid receptor agonist amounts is envisioned (i.e., each possible permutation of these amounts is envisioned). Moreover, each such permutation is envisioned wherein the nicotinic receptor agonist is nicotine and the opioid receptor agonist is morphine.

In another embodiment of the first method, the subject is a mammal. In another embdiment, the mammal is a human. In another embodiment the subject is male or female. In a further embodiment, the subject is a smoker or non-smoker.

In another embodiment of the first method, the pain is acute pain or chronic pain.

In a further embodiment of the first method, the nicotinic receptor agonist is selected from the group consisting of nicotine, meta-nicotine, DMBX-anabaseine, anabaseine, choline, acetylcholine, epibatidine and cytisine. In the preferred embodiment, the nicotinic receptor agonist is nicotine.

In another embodiment of the first method, the opioid receptor agonist is selected from the group consisting of morphine, meperidine, fentanyl, hydromorphone, alfentanil, remifentanil, carfenanil, sufenanil, butorphanol, buprenorphine and pentazocine. In the preferred embodiment, the opioid receptor agonist is morphine.

In another embodiment of the first method, the nicotinic receptor agonist is nicotine and the opioid receptor agonist is morphine.

In another embodiment of the first method, the nicotinic receptor agonist is administered orally, intranasally, transdermally, epidurally, intrathecally or intravenously.

In another embodiment of the first method, the opioid receptor agonist is administered orally, intranasally, transdermally, epidurally, intrathecally or intravenously.

In another embodiment of the first method, the nicotinic receptor agonist is administered via a single dose. In another embodiment, the nicotinic receptor agonist is administered via a plurality of doses.

In another embodiment of the first method, the opioid receptor agonist is administered via a single dose. In another embodiment, the opioid receptor agonist is administered via a plurality of doses.

In another embodiment of the first method, the nicotinic receptor agonist and the opioid receptor agonist are each administered via a plurality of doses. In another embodiment, the nicotinic receptor agonist and the opioid receptor agonist are each administered via a single dose. In a further embodiment, the single dose is administered over a period of time. In another embodiment, the period of time is at least one hour and the administration is intravenous. In another embodment, the nicotinic receptor agonist and the opioid receptor agonist are administered separately. In another embodiment, the nicotinic receptor agonist and the opioid receptor agonist are administered together as a single composition. In one embodiment, the single composition is administered intravenously. In another embodiment, the single composition is administered transdermally.

In one embodiment of the first method, the nicotinic receptor agonist is administered while the subject is conscious or unconscious. In another embodiment, the opioid receptor agonist is administered while the subject is conscious or unconscious.

This invention provides a composition comprising a pharmaceutically acceptable carrier, a nicotinic receptor agonist and an opioid receptor agonist, wherein the nicotinic receptor agonist and the opioid receptor agonist are present in a ratio of between greater than 1:100 and less than 3:4.

In one embodiment of the composition, the ratio of nicotinic receptor agonist and opioid receptor agonist is between 1:10 and 1:3. In another embodiment, the ratio of nicotinic receptor agonist to opioid receptor agonist is 1:4. In a further embodiment, the ratio of nicotinic receptor agonist to opioid receptor agonist is 1:8.

This invention provides a transdermal patch comprising a nicotinic receptor agonist and an opioid receptor agonist, whereby the nicotinic receptor agonist and opioid receptor agonist are released into a subject upon placing the patch on the subject's skin. In one embodiment, (a) the nicotinic receptor agonist is nicotine, (b) the opioid receptor agonist is morphine, and (c) the nicotinic receptor agonist and the opioid receptor agonist are present in a ratio of about 1:8.

This invention provides an article of manufacture for the intravenous administration of a composition to a subject comprising a packaging material having therein a composition comprising (a) a pharmaceutically acceptable carrier suitable for intravenous administration, (b) a nicotinic receptor agonist and (c) an opioid receptor agonist, wherein the nicotinic receptor agonist and the opioid receptor agonist are present in a ratio of 1:8. In one embodiment, the nicotinic receptor agonist is nicotine, and the opioid receptor agonist is morphine.

This invention provides a second composition comprising a nicotinic receptor agonist and an opioid receptor agonist.

In this invention, “administering” the agonists can be effected or performed using any of the various methods and delivery systems known to those skilled in the art. The administering can be performed, for example, intravenously, orally, nasally, via implant, transmucosally, transdermally, intramuscularly, intrathecally, epidurally and subcutaneously. The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

In one embodiment, the effective amount of nicotine, administered transdermally, is a dosage determined based on dosages used in commercially available nicotine transdermal patches (e.g., NICOTROL® (Pharmacia & Upjohn Co.), as described in U.S. Pat. No. 5,593,684 or 5,721,257).

All embodiments of the first method for reducing, or inhibiting the onset of, pain are envisioned mutatis mutandis, as applicable, with respect to the second method for reducing, or inhibiting the onset of, pain and the instant compositions and article of manufacture.

This instant invention is illustrated in the Experimental Details section that follows. This section is set forth to aid in an understanding of the instant invention but is not intended to, and should not be construed to, limit in any way the invention as set forth in the claims which follow thereafter.

Experimental Details

We demonstrated profound synergy between nicotine and opioids in an animal model of incisional pain, and followed this up with a double blinded, randomized, clinical study of 20 non-smoking women after gynecological surgery. The clinical study demonstrated a highly statistically significant reduction in peak pain scores (VAS reduced from 8 to 5 (out of 10)). The reduction in VAS pain score was accompanied by a 50% reduction in morphine requirement in the first hour (Flood and Daniel, 2004).

### First Series of Experiments

A. Clinical Study (Methods)

This study was a randomized, double blind clinical trial with a 24 hour data collection period. The study was approved by the institutional review board at Columbia University. Written informed consent was obtained from all patients. Women between the ages of 18 and 50, scheduled to have uterine surgery (either myomectomy or hysterectomy) through a low transverse incision were eligible to be included. We chose to only study women in this preliminary study because animal studies have identified gender differences in nicotine's analgesic action 10-12. Patients were excluded who had smoked during the past year, had preexisting pain syndromes, hypertension, or any history of cardiovascular disease. All patients were American Society of Anesthesiologists class 1 or 2. The patients were instructed before surgery that they would be asked about their pain with a numerical analog score where 0 is no pain and 10 is the worst pain that they could imagine. All patients had access to morphine with patient controlled analgesia (PCA) after surgery and were instructed in its use in the preoperative period. No other medications were given in the preoperative period.

All patients were given a standardized anesthetic as follows: anesthetic premedication was administered in the operating room consisting of 1-2 μg/kg fentanyl and 1 mg vecuronium. Anesthesia was induced with propofol 2 mg/kg and succinylcholine 2 mg/kg. After intubation of the trachea, anesthesia was maintained with a fentanyl infusion of 1-2 μg/kg/hr and isoflurane titrated to adequate anesthetic depth by the clinical anesthesiologist, who was not aware of the treatment group. Muscle relaxation was effected with vecuronium. All patients were given dolasetron 12.5 mg prophylactically to prevent nausea and vomiting. At the completion of surgery, the anesthesiologist was given an opaque sealed container with either a nicotine nasal spray (3 mg—Nicotrol NS, Pharmacia, Peapack, N.J.) or saline nasal spray prepared according to a random number table by the research pharmacy. The study drug was administered by the anesthesiologist as 3 jets in each nostril (3 mg nicotine or an equal volume of saline) at a 45 degree angle after the muscle relaxant was reversed and while the surgeon was closing the fascia.

Five minutes after extubation and every 5 minutes thereafter for 60 minutes, the patient was asked to report the pain that they were experiencing with a numerical analog score from 0-10. A pain report was also obtained at 2 and 24 hours. A PCA pump was inserted into the patient's intravenous circuit prior to emergence from anesthesia. It was programmed to deliver a dose of 1 mg morphine when the button was pressed with a lockout interval of 6 minutes and a maximal dose during one hour of 10 mg. The PCA pump was also programmed to allow a rescue dose of 3 mg morphine to be administered by a nurse every 5 minutes with additional 12 mg morphine maximally by this route every 4 hours, if the patient's pain score was greater than 3/10. As such a patient could receive 24 mg of morphine as a maximal dose in the first hour. According to our institution's standard PCA orders, the rescue dose was not administered by the nurse if the patient had a respiratory rate less than 8 breaths per minute or was determined by the nurse to be over sedated (sedation scale≦3 where 0-reflexes not present, 1-reflexes present, does not respond to verbal command, 2-eyes open to verbal command or response to name, 3-lightly asleep, eyes open intermittently, 4-fully awake, conversant). No other postoperative analgesic medications were used.

Blood pressure was measured with a non-invasive automatic oscillometric blood pressure cuff every 5 minutes for the first hour (Agilent Technologies, Andover, Mass.). Heart rate was monitored continuously with a pulse oximeter and electrocardiographic leads and recorded every five minutes for the first hour (Agilent Technologies, Andover, Mass.). All testing was conducted by the same investigator (D.D.). Both the investigator recording data and the nurse administering medication were blinded to treatment group.

The data were analyzed per protocol. Two subjects from whom informed consent was obtained were not studied for postoperative pain. In one case because of a protocol violation in the standard anesthetic the patient was not randomized and in the second case, the anesthesiologist was not certain of study drug dose. The decision to exclude these patients was made prior to the postoperative period and this no data was obtained.

Statistical Analysis

In previous studies using analog pain scores the standard deviation was approximately 2 numerical analog pain units. We calculated that in order to detect a difference of 2 numerical analog pain units, with α=0.05 and 80% power we would need to enroll 17 patients per group (StatMate, GraphPad software, San Diego, Calif., USA). 10 patients were enrolled per group as a pilot study requested by our IRB. The difference between groups in pain score, morphine utilization and hemodynamic variables were compared with a One Way Analysis of Variance. The change in pain scores over time was determined with a One Way Analysis of Variance for repeated measures, where time was considered a continuous variable (Analyse-it for Microsoft Excel; Analyse-it Software, ltd., Leeds, England). All values are reported as mean (SD).

B. Results

The patients did not differ in age, weight or duration of surgery. Of note, there was no difference in intraoperative fentanyl utilization between groups (Table 2).

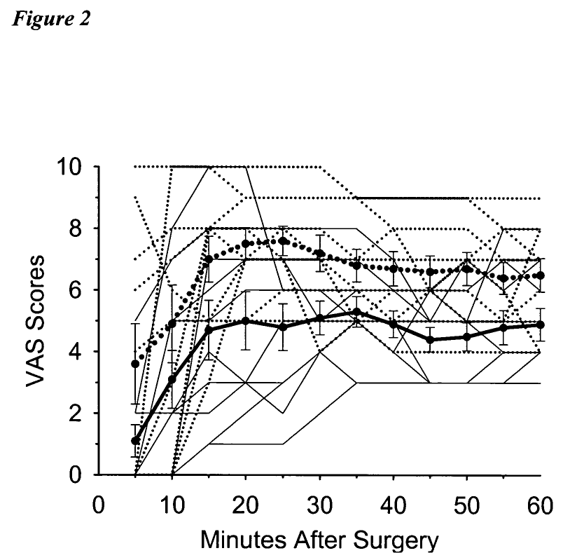

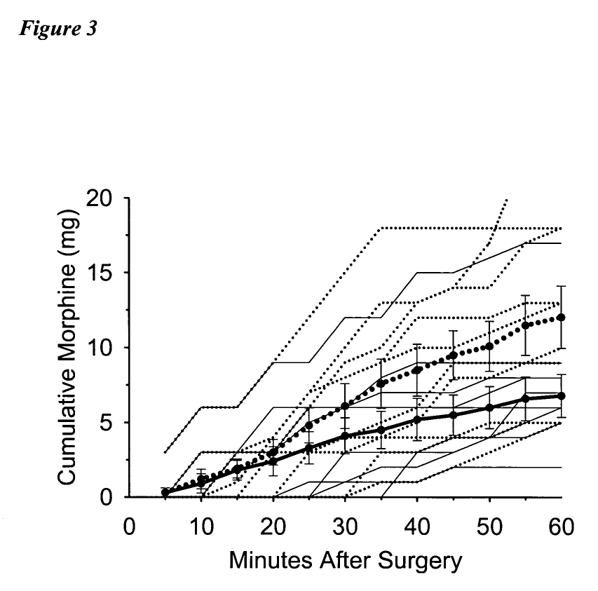

Patients in the placebo group had considerable postoperative pain. Despite treatment with morphine, the peak pain score in the placebo group was 7.6 (1.5) units and occurred 25 minutes after extubation of the trachea (zero time point, FIG. 2). Thereafter, pain scores in the placebo group were significantly reduced to a minimum of 6.4 (1.4) at 55 minutes after emergence (P<0.01). The mean dose of PCA morphine used by the control group was 12 (6) mg in the first hour (FIG. 3).

In contrast, patients who were treated with nicotine nasal spray just prior to emergence from the isoflurane anesthetic reported lower pain scores at all times during the first hour than patients who received placebo (FIG. 2, P<0.001). The peak pain score in the nicotine group was 5.3 (1.6) units and occurred 35 minutes after surgery. Both groups reported increasing pain scores in the first 20 minutes after emergence when the effect of the fentanyl administered intraoperatively would be expected to be dissipating. Twenty-four hours after emergence from the general anesthetic, the patients in the nicotine group still reported lower pain scores than those in the placebo group (4.9 (1.4)-placebo, 1.5 (0.5)-nicotine; P<0.01). In conjunction with decreased pain experience in the nicotine group, cumulative morphine utilization was less in the nicotine group than the placebo group during the first hour with 12 (6) mg used by the control group compared to 6 (5) mg used by the nicotine group (FIG. 3, P<0.05). During the first 24 hours after surgery, the patients who received placebo used 63±11 mg of morphine and the patients in the nicotine group used 41±9 mg morphine. The difference in morphine utilization at 24 hours did not reach statistical significance.

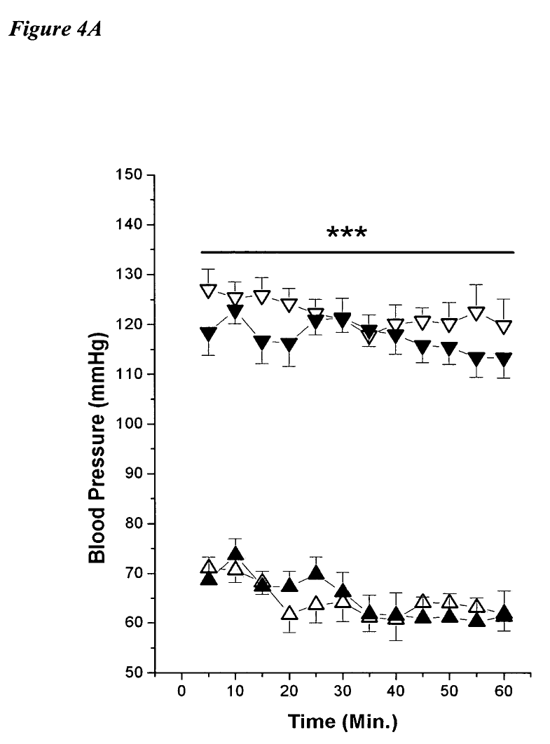

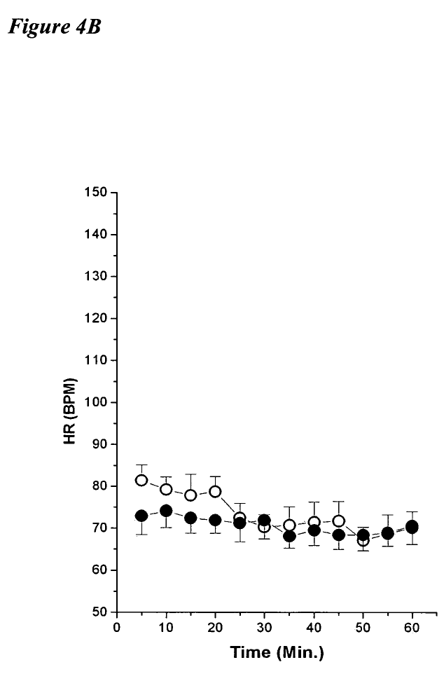

Because nicotine nasal spray can cause increases in blood pressure and heart rate when used by unanesthetized subjects 14-18, we measured these hemodynamic variables over the first hour in both groups. Systolic blood pressure was actually lower in the nicotine group, possibly associated with their lower reported pain scores (FIG. 4A; 105 (3) vs. 122 (3); P<0.001). There was no difference in heart rate or diastolic blood pressure over the first postoperative hour (FIG. 4B).

In sum, throughout the first hour after surgery, the patients treated with nicotine reported lower pain scores. Repeated measures ANOVA with NONMEM demonstrated that the nicotine effect was statistically significant (p<0.01) at all points in time. We observed decreased VAS scores in the nicotine group at 24 hours, and specifically tested with NONMEM whether this was statistically significant as well. It was; which suggests a persistence of drug effect well beyond nicotine's known short duration of action.

FIG. 3 shows morphine utilization in the patients who received nicotine (solid lines) or placebo (dotted lines). Analysis with NONMEM demonstrated that the decreased morphine utilization was statistically significant (p<0.01). Overall, morphine usage was about half in the nicotine group compared to the placebo group. Systolic blood pressure was lower in the group that received nicotine (105 (3) vs. 122 (3); P<0.001), but there was no difference in diastolic blood pressure or heart rate. The incidence of nausea and vomiting did not differ and the women did not differ in any demographic variables.

C. Discussion

Our study unambiguously demonstrates that nicotine potentiates opioid analgesia in the early post-operative period. We were surprised at the level of statistical significance (p<=0.01 for both primary endpoints) despite the small number of subjects. We performed a NONMEM analysis of subsets to identify whether or not this high level of significance was related to a single outlying subject. It was not.

This study raises several intriguing questions. First, why didn't the patients in the placebo group just receive more morphine to get to the same level of analgesia as patients receiving nicotine? This pinpoints the fundamental problem of opioid analgesia for acute post-operative pain: there may be no dose of opioid that provides adequate analgesia for acute pain in the awake patient without exposing that same patient to risk of airway obstruction and ventilatory depression when that patient falls asleep. Patients and clinicians both tolerate inadequate pain control to limit the ventilatory depression, sedation, nausea, vomiting, pruritis, and ileus associated with opioid use. In this study, patients in both groups received the standard-of-care titration of opioid analgesia available to post-operative patients at Columbia University. The poor pain control seen in the placebo group receiving standard-of-care pain management at Columbia University reproduces the statistic quoted above that “seventy to eighty percent” of post-operative patients have poorly managed pain. To be blunt, we rely on opioids for post-operative analgesia, and as a result we don't manage post-operative pain well. The quoted statistics suggest nobody else does much better. The solution is not to find a magic dose of morphine that will somehow make patients comfortable without toxicity, but instead to improve our pharmacological approaches to pain management.

What is difficult to communicate in a graph, but was obvious in the study despite rigid blinding, is that patients receiving nicotine had qualitatively different, and better, analgesia. During the study some patients commented on being surprisingly comfortable, while others were clearly inadequately managed. Two patients wrote unsolicited letters to the investigator thanking her for being in the study, because they had so little pain. When the study blind was broken, both patients who wrote letters, as well as the ones who had commented during the study about how little pain they were experiencing, were in the nicotine group.

While these data demonstrate that nicotine potentiates morphine analgesia, it is less clear whether this is an additive effect, pharmacological synergy, an effect on tolerance, or possibly a pharmacokinetic interaction. If the interaction is pharmacodynamic, it likely represents true synergy, rather than additive drug effect, because of the low potency of nicotine as an analgesic, and because the rapid half-life of nicotine precludes sustained analgesic effect as was observed in this study. It is also possible that nicotine delayed the onset of morphine tolerance. This could account for a sustained benefit at 24 hours, but is unlikely to be associated with the benefit in the first hour. Although pharmacokinetic mechanisms could also be relevant, particularly since both nicotine and morphine are metabolized by glucuronidation in the liver, the quantitative and qualitative differences in analgesia despite nearly unrestricted access to morphine make it unlikely that the observed effect could be explained simply by more system morphine available in the nicotine groups.

The duration of nicotine's analgesic effect was at least 24 hours (the last assessment in the study). Although the terminal half life of nicotine is often described as just 2-3 hours, ultra low concentrations are present much longer due to release from less vascular tissue stores. (Sutherland, Russell et al. 1992; Schneider, Lunell et al. 1996; Benowitz, Zevin et al. 1997; Guthrie, Zubieta et al. 1999; Fishbein, O'Brien et al. 2000). The prolonged effect of a single dose of nicotine might be due to the continued presence of low concentrations of nicotine and synergy with PCA morphine. Alternatively, the persistence of analgesic effect to the 24 hour time point could be due to an early effect of nicotine on synaptic plasticity, preventing central or peripheral sensitization to the pain stimulus. As mentioned above, this could also be a result of nicotine delaying the onset of tolerance to morphine. It could reflect altered perceptions of overall analgesic adequacy in patients spared the severity of the first hours of acute postoperative pain.

It is also possible that the analgesic effects of nicotine seen in our human study could be mediated through an anti-inflammatory mechanism. Nicotine has anti-inflammatory action through modulation of peripheral macrophages in the periphery and microglia in the CNS though activation of α7-nicotinic receptors (Wang, Yu et al. 2003; Miao, Green et al. 2004; Shytle, Mori et al. 2004). The nervous system, through vagus nerve activity and acetylcholine activation of α7-nicotinic receptors on macrophages, can inhibit the release of the pro-inflammatory cytokine TNF-α, and thus attenuate systemic inflammatory responses (Wang, Yu et al. 2003). Activation of α7-nicotinic receptors on CNS microglia results in the activation of a similar anti-inflammatory pathway (Shytle, Mori et al. 2004). Because post-operative pain, unlike most common experimental pain paradigms has a component of inflammation and wound healing, the prolonged analgesic action of nicotine in our study could be a result of anti-inflammatory effects of nicotine.

TABLE 2
Demographic Variables and Intraoperative Fentanyl Use

| Variable | Placebo | Nicotine |
|---|---|---|
| Age (yr) | 46 ± 2 | 43 ± 3 |
| Weight (kg) | 67 ± 4 | 65 ± 5 |
| Duration of Surgery (min) | 147 ± 19 | 124 ± 9 |
| Intraoperative | 2.3 ± 0.5 | 2.1 ± 0.2 |
| Fentanyl dose (μg/min) | | |

There was no difference in age, weight, duration of surgery or dose of narcotic used during surgery between groups.
Values are mean ± SD.

### Second Series of Experiments

I. Introduction

We have chosen to study pain sensitivity in a mouse model of post-incisional pain, because pain after surgery is likely to be different and more complex then simpler tests of heat and pressure sensitivity in uninjured animals. Indeed, different pain testing paradigms have yielded different results, particularly with respect to nicotinic pharmacology (Caggiula, Epstein et al. 1995). Our model was developed from that of Brennan in rats (Brennan, Vandermeulen et al. 1996) and modified for mice by Pogatzki (Pogatzki and Raja 2003). It is described in detail in the methods which follow. We have chosen mice as an experimental animal because of the ability to extend findings into pharmacogenomics, make use of genetically modified animals and in order to relate to our previous research and control studies.

Briefly, an incision is made in the mouse's hind paw under isoflurane anesthesia, the muscle layer is disrupted, and the wound is sutured closed. Two hours after recovery from anesthesia, the area immediately surrounding the wound is probed with von Frey fibers, and the threshold to evoke movement of the injured paw is measured. The upper threshold is 15 grams (the animal only weighs 25 grams). The wound may also be probed with a heat source. Both assays may be completed on one animal in order to gain maximal information from the experiment and to draw out differences that may be due to underlying differences in neurobiology.

II. Materials and Methods

A. Animals

With the approval of the animal use and care committee at Columbia University, female 129J mice (Jackson Laboratories, Bar Harbor, Me.) at 6 to 10 weeks of age were used. They were housed in groups of 5 and had free access to food and water. Animals were housed in an American Association of Laboratory Animal Care-approved facility. At the end of the experiments, all mice were euthanized with CO₂.

B. Surgical Procedure—Post Operative Pain Model

Mice were anesthetized with 1.5% to 2.5% isoflurane in oxygen until there was no response to a paw and tail pinch. Alcohol 70% was swabbed on the foot before the surgery began as an antiseptic measure. Next a 5 mm longitudinal incision was made with a no. 15 blade through the skin and fascia of the plantar foot. The incision started 2 mm from the proximal edge of the heel and extended toward the toes. The underlying muscle was elevated with forceps, leaving the muscle origin and insertion intact. Finally the skin was apposed using a single polysorb suture, and the wound covered with an antibiotic ointment. The mice were then allowed a 2 hour recovery period before behavioral testing began.

C. Behavioral Testing

1. Hargraves

We measured hind paw withdrawal latency in up to five unrestrained mice (per study) housed individually in clear plastic chambers. The chambers rested on a clear glass plate. The glass plate was warmed to minimize body heat loss. To diminish exploratory activity, the mice were acclimated to this environment for at least 30 minutes before commencing the study. After acclimation, a movable source of radiant heat was applied from a lamp through an aperture under the glass plate to the hind paw of the resting mouse. The testing stimulus was 15% of maximal that caused an average increase to 42° C. on movement. An investigator measured the time from the onset of the application of the light (heat) to the time the mouse moved the hind limb.

2. von Frey

The mice were placed on an elevated mesh floor and enclosed in clear plastic chambers. Again, to reduce exploratory activity, the mice were allowed to acclimate to this environment for approximately 30 minutes before testing. von Frey filaments (Pogatzki et al. 2003) were pushed up through the mesh flooring and against each mouse's hind paw. Each von Frey filament is calibrated to a specific value of grams of bending force. A response to a filament was identified as the withdrawal of the paw when pressure was applied for 1 second. The von Frey filaments were applied in order of increasing pressure until a paw withdrawal took place. Then the filament's grams of bending force value and that of the previous filament were averaged giving us the final value of bending force that could be tolerated by that individual mouse. These tests were performed multiple times to each paw to ensure accuracy. Lastly, before analysis, the grams of bending force values were converted into milli-Newtons (mN).

II. Results and Discussion

Two hours after surgery, the baseline hyperalgesic response was measured. Animals then received nicotine and/or morphine by IP injection. We analyzed the morphine-nicotine analgesic interaction using the response surface methodology of Minto and implemented in NONMEM. (Minto, Schnider et al. 2000).

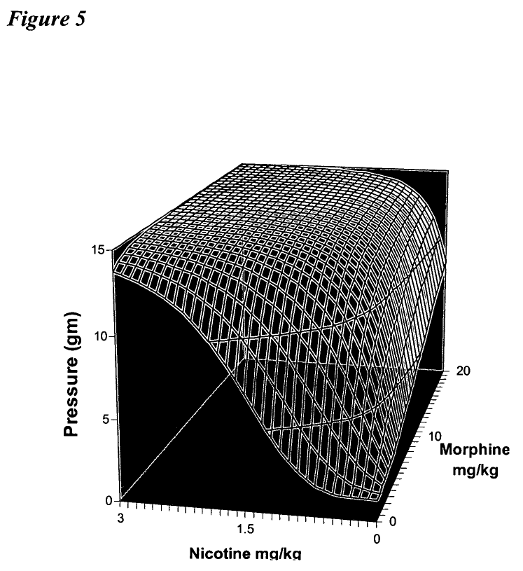

FIG. 5 shows the response surface for morphine—nicotine analgesic interaction (data presented in Table 3). The interaction was highly synergistic (p<<0.01). The mouse strain used (129J-Jackson laboratories, Bar Harbor, Me.) is relatively insensitive to morphine and our range was limited by the hyper-aesthetic response induced by opioid agonists in mice. Although morphine was relatively ineffective alone, the addition of a small dose of nicotine greatly increased the pressure that the animal could tolerate around the wound. These data demonstrate that the antinociceptive action of morphine and nicotine is highly synergistic in this mouse model.

We examined the influence of nicotine on the time course of the development of morphine tolerance by subcutaneously inserting a mini-osmotic “Alzet” infusion pump (Alza Corporation, Mountain View, Calif.) designed to administer morphine 2 mg/kg/hr. The animals then had a hind paw incision, as described above, and received 1 mg/kg IP nicotine or saline. The investigator was blinded to treatment group. The area immediately surrounding the wound was probed with a heat stimulus and the latency (seconds) to withdrawal of the injured paw was recorded.

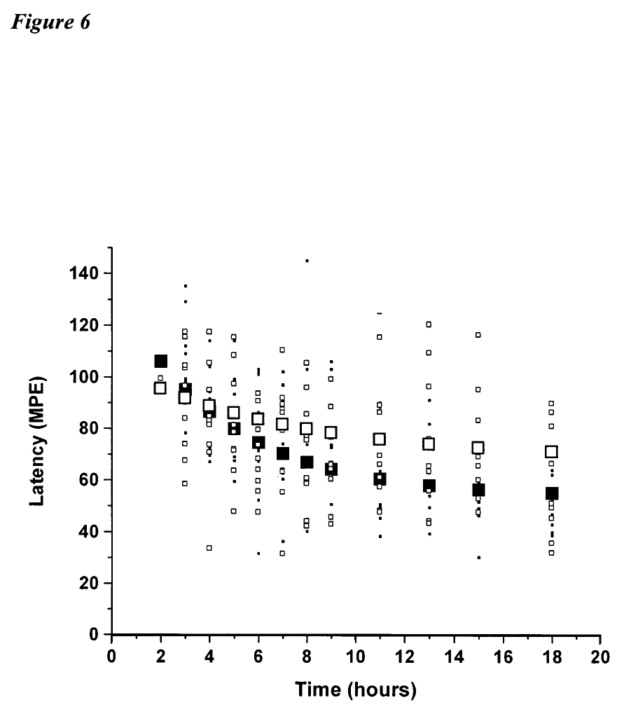

Peak latency was higher in the nicotine group (data not shown) as would be expected from the experiments above. FIG. 6 shows the change from maximal withdrawal latency over an 18 hour period. The animals developed significant tolerance to morphine that is well described using a mono-exponential decay function. Animals treated with nicotine and morphine had a plateau in their analgesic action at a significantly higher level than those that were treated with only morphine (30±6% vs. 64±14% reduction in peak effect).

TABLE 3

| Morphine | Nicotine | Meas | Morphine | Nicotine | Meas | Morphine | Nicotine | Meas |
|---|---|---|---|---|---|---|---|---|
| (mg/kg) | (mg) | (gms) | (mg/kg) | (mg) | (gms) | (mg/kg) | (mg) | (gms) |
| 13 | 1.5 | 15 | 15 | 0 | 7.9 | 0 | 1 | 1.95 |
| 13 | 1.5 | 15 | 15 | 0 | 4.9 | 0 | 1 | 2.7 |
| 13 | 1.5 | 15 | 0.1 | 0 | 2.95 | 0 | 1 | 2.7 |
| 13 | 1.5 | 15 | 0.1 | 0 | 0.75 | 0 | 1 | 2.7 |
| 13 | 1.5 | 15 | 0.1 | 0 | 1.8 | 0 | 1 | 1.95 |
| 13 | 1.5 | 15 | 0.1 | 0 | 1.8 | 0 | 0.5 | 0 |
| 13 | 1.5 | 15 | 0.1 | 0 | 0.75 | 0 | 0.5 | 0 |
| 13 | 1.5 | 15 | 20 | 0 | 7.9 | 0 | 0.5 | 0 |
| 13 | 1.5 | 15 | 20 | 0 | 4.9 | 0 | 0.5 | 0.25 |
| 13 | 1.5 | 15 | 20 | 0 | 8.1 | 0 | 0.5 | 0 |
| 10 | 1.5 | 15 | 20 | 0 | 4.9 | 0 | 3 | 15 |
| 10 | 1.5 | 15 | 20 | 0 | 4.9 | 0 | 3 | 15 |
| 10 | 1.5 | 15 | 1 | 0 | 1.95 | 0 | 3 | 15 |
| 10 | 1.5 | 15 | 1 | 0 | 1.7 | 0 | 3 | 15 |
| 10 | 1.5 | 15 | 1 | 0 | 1.95 | 0 | 3 | 15 |
| 1 | 1.5 | 13.65 | 1 | 0 | 0.9 | 0 | 5 | 15 |
| 1 | 1.5 | 13.65 | 1 | 0 | 1.95 | 0 | 5 | 15 |
| 1 | 1.5 | 15 | 10 | 0 | 1.1 | 0 | 5 | 15 |
| 1 | 1.5 | 13.65 | 10 | 0 | 1.1 | 0 | 5 | 15 |
| 1 | 1.5 | 13.65 | 10 | 0 | 0.45 | 0 | 5 | 15 |
| 0.1 | 1.5 | 13.65 | 10 | 0 | 2.15 | 0 | 1.5 | 2.7 |
| 0.1 | 1.5 | 13.65 | 10 | 0 | 1.1 | 0 | 1.5 | 2.7 |
| 0.1 | 1.5 | 7.65 | 5 | 0 | 0.9 | 0 | 1.5 | 4.9 |
| 0.1 | 1.5 | 7.9 | 5 | 0 | 0.9 | 0 | 1.5 | 4.65 |
| 0.1 | 1.5 | 13 | 5 | 0 | 1.1 | 0 | 1.5 | 4.9 |
| 13 | 0.5 | 15 | 5 | 0 | 1.1 | 13 | 5 | 15 |
| 13 | 0.5 | 15 | 5 | 0 | 0.9 | 13 | 5 | 15 |
| 13 | 0.5 | 13.9 | 5 | 0 | 7.9 | 13 | 5 | 15 |
| 13 | 0.5 | 13.65 | 5 | 0 | 7.9 | 13 | 5 | 15 |
| 13 | 0.5 | 13.65 | 5 | 0 | 7.65 | 20 | 1.5 | 15 |
| 13 | 1 | 15 | 5 | 0 | 7.9 | 20 | 1.5 | 15 |
| 13 | 1 | 15 | 5 | 0 | 7.65 | 20 | 1.5 | 15 |
| 13 | 1 | 15 | 1 | 0 | 5.2 | 20 | 1.5 | 15 |
| 13 | 1 | 15 | 1 | 0 | 5.95 | 20 | 1.5 | 15 |
| 13 | 1 | 15 | 1 | 0 | 0.75 | | | |
| 5 | 1.5 | 15 | 1 | 0 | 5.2 | | | |
| 5 | 1.5 | 15 | 1 | 0 | 2.95 | | | |
| 5 | 1.5 | 15 | 13 | 0 | 4.65 | | | |
| 5 | 1.5 | 15 | 13 | 0 | 4.65 | | | |
| 5 | 1.5 | 15 | 13 | 0 | 7.65 | | | |
| 5 | 1.5 | 15 | 13 | 0 | 7.65 | | | |
| 5 | 1.5 | 15 | 13 | 0 | 7.65 | | | |
| 5 | 1.5 | 15 | 0 | 2 | 2.7 | | | |
| 5 | 1.5 | 15 | 0 | 2 | 4.9 | | | |
| 15 | 0 | 7.9 | 0 | 2 | 2.7 | | | |
| 15 | 0 | 4.9 | 0 | 2 | 2.7 | | | |
| 15 | 0 | 7.9 | 0 | 2 | 4.9 | | | |

### Planned Research Design and Methods

Based on the data set forth in the First and Second Experimental Series above, we hypothesize that:
1. The rodent model will be useful to understand the mechanisms of nicotinic analgesia for postoperative pain. Specifically, it will help us identify issues of optimal timing of drugs, persistence of drug effect, differentiation between decreased morphine tolerance and increased morphine efficacy as well as possible pharmacokinetic interactions.
2. The effectiveness of nicotine in the clinical setting is due to the synergistic interaction between the nicotine (acting on α4β2 containing nicotinic receptors) and the opioid agonist.
3. Nicotine has long-lasting anti-inflammatory action (due to activation of α7 containing receptors on macrophages) that adds to its analgesic effects.
4. The prolonged analgesic effect of a single dose of nicotine is due to decreased tolerance to opioids.
5. Nicotine will have analgesic efficacy in men and women, smokers and non-smokers.

Following the translational paradigm in the First and Second Series of Experiments set forth above, the following experiments propose to further characterize and, where possible, identify mechanisms that mediate the nicotinic-morphine antinociception relationship in a murine model of postoperative pain, and to use those results to guide clinical research on nicotine-morphine interaction. The animal studies outlined below will:
1. Further refine the nicotine morphine drug interaction surface.
2. Determine the time course (concurrent with opioid or before opioid) of nicotine administration.
3. Determine the relevant nicotinic subtype for antinociceptive synergy with opioids.
4. Determine the effect of nicotine on the development of tolerance to opioid analgesia.
5. Determine the effect of nicotine on inflammatory mediators and physical signs of inflammation in the rodent and determine if these are correlated with analgesia.

We have proposed two related clinical studies of nicotinic antinociception in different surgical settings. The first proposed clinical study, Specific Aim 2, is an expansion of our pilot study on the effect of a single dose of nicotine nasal spray in women undergoing gynecological surgery will allow us to:
1. Confirm and extend the results of our pilot study.
2. Confirm the persistence of nicotine effect at 24 hours.
3. Determine the influence of anesthetic choices (isoflurane vs. propofol) influences the nicotine-morphine analgesic response. (Because nicotine reverses isoflurane hyperalgesia (Flood, Sonner et al. 2002), this question will help determine the overall clinical significance of the nicotine-morphine analgesic response).

The second proposed clinical study, Specific Aim 3, is in patients having third molar extractions. In this setting, limited to patients in whom all 4 third molars are extracted, patients have two operations (each of which removes two molars) separated by several weeks. This is a unique and potentially powerful study design, as each subject will be crossed over from nicotine to placebo or vice versa, thus acting as his or her own control. We intend to use the power of a cross-over design to identify the role of several important factors:
1. Compare nicotinic analgesia between men and women
2. Compare nicotinic analgesia between smokers and non-smokers
3. Follow patient's pain for a longer period of time to determine how long the benefit of nicotine lasts.
4. Compare the effect of nicotine on the anti-inflammatory marker TNF-α and to relate those changes to pain relief.
5. Extend our findings to a different type of surgical procedure.

Based on our results, we will be able to conclude whether there is evidence for synergy with activation of m-opioid receptors and a particular subset of nicotinic receptors. We will also know whether changes in tolerance and inflammation are likely to be involved in the remarkable, long-lasting analgesic effect of nicotine that we found in our clinical study (Flood and Daniel 2004). Furthermore, we will know whether nicotinic analgesia is specific to patients after a volatile anesthetic (due to the pronociceptive effects of low concentrations of anesthetic gases) or is more generalizable. We will know whether nicotine is an effective analgesic adjuvant in men and smokers as well. In short, these clinical studies will identify whether the intriguing findings in our pilot data are clinically generalizable, or are a curiosity limited to a small subset of patients.

Specific Aim 1: To determine putative mechanisms and determinants (synergistic interactions between nicotinic agonists and opioids, the effect of nicotine on opioid tolerance, inflammatory modulation and sensitization) involved in the antinociceptive effect of nicotine using a mouse model of post-operative pain.

Rationale and Significance: In this aim, we will detect the specific nicotinic subtype that mediates the synergy with morphine that we have demonstrated in our data (FIG. 5). In this aim we will also study the effect of nicotine on morphine tolerance and postoperative inflammation, important issues in pain sensitivity. Lastly, we will examine the timing and administration of nicotine relative to morphine and the surgical stimulus to help guide subsequent human clinical trials.

We will use an animal model for these mechanistic studies because we can use a wide range of drug doses in a model surgical paradigm, as well as study experimental nicotinic ligands. We strongly favor murine models, rather than rat models, because of the availability of knock-out mice for particular receptor subtypes.

The experiments proposed in this aim all utilize a model of acute incisional pain developed by Brennan in rats (Brennan, Vandermeulen et al. 1996) and modified by Pogatzki for use in mice (Pogatzki and Raja 2003). Although there are several useful methods for assessing pain and analgesia, there is some evidence that post surgical pain is unique in its etiology, effects on plasticity and the neurotransmitter systems involved (Brennan 2002). These considerations are critically important for the study of nicotinic analgesia, as different study paradigms have produced different results. We believe that the Brennan-Pogatzki model mimics the clinical situation well, which facilitates translating the animal results into clinical trials. The injury induced and the resulting allodynia is reproducible and stable over 4-5 days (Pogatzki and Raja 2003). These animals are commercially available through Jackson Laboratories (Bar Harbor, Me.) and through collaboration.

There are 5 experimental studies in this specific aim:
1. The first experimental series will identify the nicotinic subtype responsible for synergy with morphine. This step is important because there are many subtype selective nicotinic agonists currently in clinical trials for other indications and improved efficacy or safety may be found with more specific drugs.
2. The second experimental series will further examine nicotine's effect on tolerance to morphine. If an effect on tolerance is unambiguously demonstrated, we will determine the nicotine subtype responsible for synergy with morphine.
3. In experimental series 3 we will determine the effect of nicotine on the inflammatory mediators TNF-α, IL2 and IL6. These cytokines have been implicated in the anti-inflammatory properties of nicotine (Wang, Yu et al. 2003). Similar experiments will be proposed in aim 3 in our clinical study to corroborate the animal results.
4. In experimental series 4, we will determine the effect of nicotine treatment on the area of secondary hyperalgesia. This measure has been correlated with the degree of neuronal sensitization (Zahn and Brennan 1999).
5. In experimental series 5, we will determine whether the timing of nicotine (before vs. after incision) and the method of delivery (IP injection vs. continuous subcutaneous infusion) affects the interaction with morphine.

Experimental Series 1: Synergy between μ-opioid and Nicotinic Activation

Our preliminary animal studies (FIG. 5) demonstrate significant antinociceptive synergy when nicotine and morphine are used together. Nicotine is the prototypical agonist that activates a broad spectrum of nicotinic receptors including heteromeric α4β2 subunit containing receptors, α3β4. subunit containing receptors, and the lower affinity α7 subunit containing nicotinic receptors. Most nicotinic receptors in the CNS contain α4 and β2 subunits and nicotinic receptors expressing these receptors are thought to play a significant role in nicotinic analgesia (Damaj, Glassco et al. 1999; Marubio, del Mar Arroyo-Jimenez et al. 1999; Bitner, Nikkel et al. 2000; Jain 2004). There is some evidence that nicotinic receptors containing the α7-subunit play a role in nicotinic antinociception (Damaj, Meyer et al. 2000), and α7-containing nicotinic receptors clearly play a special role in neuronal plasticity (Colquhoun and Patrick 1997). Also, α7 containing nicotinic receptors modulate release at capsaicin sensitive primary afferent neurons (Roberts, Stevenson et al. 1995). Activation and subsequent desensitization of α7 nicotinic receptors could be antinociceptive for heat type stimuli on this basis. Nicotinic receptors that express α3 and β4 type subunits are well known to mediate synaptic transmission in the ganglia of the autonomic nervous system, but are also expressed in anatomically localized areas of the CNS (Fu, Matta et al. 1998). Hemodynamic side effects are likely to be mediated by α3β4 containing nicotinic receptors.

In our pilot clinical study (FIGS. 2 and 3) we chose a 3 mg dose of nicotine nasal spray, because it was the high end of doses that had been used safely in human volunteers (Fishbein, O'Brien et al. 2000). Although that dose in combination with morphine improved post-operative analgesia, every subject required some morphine. As such, nicotine at near a maximum safe dosage does not have sufficient efficacy to be used as a sole agent for postoperative pain. Subtype selective nicotinic agonists that that activate either α4β2 or α7 nicotinic receptors might provide a greater dynamic range for analgesia.

Strategy: Clearly synergy with opioid agonists is a desirable characteristic. As such, we will determine whether analgesia from activation of α4β2, or α7 containing nicotinic receptors is responsible for the potent synergy that we found between nicotine and morphine. Because α3β4 nicotinic receptors represent such a small portion of CNS nicotinic receptors and mediate the hemodynamic side effects of nicotinic activation, we will not specifically target this combination.

Experiment 1a: Role of α4β2 Activation in Opioid-Nicotinic Synergy

To determine whether the synergistic interaction between morphine and nicotine is due to activation of the α4β2 containing nicotinic receptors, we will use the nicotinic agonist metanicotine that is selective for receptors containing α4 and β2 subunits. Metanicotine binds over 10,000 times more tightly to α4β2 type nicotinic receptors than to α3β4, α7 or muscle type nicotinic receptors (Bencherif, Lovette et al. 1996). As previously noted, activation of α4β2 nicotinic receptors has been implicated in most types of nicotinic antinociception (Damaj, Glassco et al. 1999; Marubio, del Mar Arroyo-Jimenez et al. 1999; Bitner, Nikkel et al. 2000; Jain 2004).

A concentration response relationship for metanicotine reduction in primary hyperalgesia after the surgical procedure will be established as we have for nicotine in the experiments for FIG. 5. In all experiments that utilize a change in primary hyperalgesia as an end point (Aim 1, Experimental series 1-3,5), we will probe immediately adjacent to the wound with both a heat stimulus (42°; latency measurement) and a pressure stimulus from a von Frey fiber (threshold measurement). Because the stimuli are mediated by different primary afferent neurons and mediated by different systems within the dorsal horn, differences in results may provide information about the neuro-physiology underlying the observed effect. Finding differences between stimulus paradigms would be used as hypothesis generating for future more specific experiments.

A concentration range surrounding a dose of 10 mg/kg will be used as this concentration causes midrange antinociception in the tail flick assay (Damaj, Fei-Yin et al. 1998). At least 5 concentrations of metanicotine will be tested to construct the concentration response relationship, from minimal to saturated effect. The time course of metanicotine will be tested with a midrange concentration. Testing will begin at the time of peak drug effect. The resulting data will be fit to a sigmoid equation and an EC₂₀ concentration for metanicotine will be determined.

The interaction between metanicotine and morphine will be studied by testing the effect of combinations of the two drugs on primary hyperalgesia as above. Specifically, the EC₂₀ concentration will be combined with morphine at each concentration used in the concentration response determined for FIG. 5 (0.1, 1, 5, 13, 15, and 20 mg/kg morphine) Similarly, the calculated EC₂₀ concentration of morphine, will be combined with at least 5 concentrations of metanicotine and the effect on primary hyperalgesia will be measured.

An interaction surface will be constructed as we have for the interaction between nicotine and morphine in FIG. 5 Excel (Microsoft, Inc, Redmond, Wash.). The data will be analyzed for significant interaction using NONMEM (Globomax, Inc., Hanover, Md.).

Experiment 1b: Role of α7 activation in Opioid-Nicotinic Synergy

To determine whether the synergistic interaction between morphine and nicotine is due to activation of the α7 containing nicotinic receptors, we will examine the interaction of morphine and the nicotinic agonist DMBX-anabaseine (also called GTS-21). DBMX-anabaseine is selective for receptors containing α7 subunits and can be used systemically because it has good CNS penetration (van Haaren, Anderson et al. 1999). It could be used with intrathecal or intracerebro-ventricular injection to confirm and extend this experiment (Damaj, Meyer et al. 2000). Mice will be treated with DMBX-anabaseine 0-8 mg/kg in to determine whether activation of α7-nicotinic receptors is anti-nociceptive in this model of postoperative pain.

The assay will use primary hyperalgesia as an endpoint as in experiment 1a. The concentrations of DMBX-anabaseine will be 0-8 mg/kg by IP injection or as needed to identify the concentration response relationship (van Haaren, Anderson et al. 1999). The data will be fit and analyzed as above.

MLA, a selective antagonist for α7, α8, α9 subunit containing nicotinic receptors, had no effect on the antinociceptive response to nicotine as measured by a tail flick assay (Damaj, Fei-Yin et al. 1998). It is possible however that activation of α7 receptors plays a role in postoperative pain. Regardless, characterization of the analgesic interaction between morphine and DMBX-anabaseine will help determine whether activation of α7 containing nicotinic receptors is required for the observed synergy. If the interaction between morphine and DMBX-anabaseine is synergistic, we would conclude that the activation of α7 subunit containing nicotinic receptors is important to opioid synergy.

Expected Results and Potential Issues

The existing data on the subunit-dependence of nicotinic analgesia is strongly in favor of a role for α4β2 containing nicotinic receptors in antinociception. As such, we anticipate that activation of α4β2 containing nicotinic receptors with metanicotine will provide analgesia that is synergistic with the prototypical μ-opioid agonist morphine.

One possible outcome is that there will be no synergy between metanicotine and morphine or DMBX-anabaseine and morphine. One interpretation of this result is that a receptor activated by nicotine, but that does not contain α4, β2 or α7 subunits, mediates the synergy between nicotine and morphine. Such a nicotinic receptor could be constructed of α3 and β4 subunits, or be a subtype that is not activated with these ligands, or even a nicotinic receptor containing subunits that have not been described. We would proceed to address the question of α3β4 CNS receptors with direct CNS injection of the selective antagonist omega-conotoxin (Kulak, McIntosh et al. 2001). There is some evidence for the existence of unknown nicotinic subunits that play a role in nicotinic antinociception (Rueter, Meyer et al. 2000).

A positive result with DMBX-anabaseine would be surprising and could be followed up with synergy experiments in α7-nicotinic WT and knockout mice (available commercially from Jackson Laboratories, Bar Harbor, Me., USA) or using acute injection of intrathecal choline. Choline, a breakdown product of acetylcholine, is also selective for α7 containing nicotinic receptors but is only effective at high concentrations (Papke, Bencherif et al. 1996; Colquhoun and Patrick 1997). Choline is less convenient than DMBX-anabaseine because it can not be used systemically as it does not cross the blood brain barrier.

Experimental Series 2: Effect of Nicotinic Activation on Opioid Tolerance

Opioids are currently the most efficacious drugs for treatment of severe pain. In chronic or long term use, tolerance to the analgesic effects of opioids is a major clinical problem. Also, the neurobiological substrates that underlie tolerance are thought to be common to those active in opioid addiction (Jasinski 1997; Inturrisi 2002; Waldhoer, Bartlett et al. 2004). It is possible that the long lasting pain benefit that we found from nicotine in our pilot clinical study might be due to a reduction in acute tolerance to morphine (Ho, Wang et al. 1999). We have addressed this issue in our animal model of post-operative pain in FIG. 6. Rodents develop acute tolerance to opioids within 24 hours of starting an infusion. We studied the development of tolerance over an 18 hour period in the setting of postoperative pain, and the effect of nicotine on the apparent tolerance to opioids. Our experiment was designed to mimic our clinical study as closely as possible. At the completion of the surgical procedure on the hind paw, a micro-osmotic pump (Alza, Mountain View, Calif.) that releases morphine at 2 mg/kg/hr was inserted subcutaneously. While still under anesthesia, the mice were injected with nicotine 1.5 mg/kg IP or saline. The investigator was blinded to the injected drug. From control experiments for experimental series 1, we know that the average latency for hind paw withdrawal when the wound is probed with heat is about 3 seconds and the threshold for withdrawal from von Frey fibers is reduced to 25 mN. In the experiments designed to determine the effect of nicotine on tolerance to morphine, the mice had near baseline thresholds after surgery with the subcutaneous morphine infusion. In mice receiving saline, significant tolerance developed over an 18 hour period. As expected, the initial analgesia was higher when the mice were treated with a single dose of nicotine rather than saline. Similar to our results in patients, the single injection of nicotine provided a long term response that could be explained by reduced tolerance to morphine (FIG. 6). In this experimental series, we will further explore the effect of nicotine on opioid tolerance and determine whether it is dependent on nicotine dose.

Experiment 2a: Time Course of Nicotinic Reduction of Opioid Tolerance.

Treatment with a single dose of nicotine reduced the tolerance that develops to morphine analgesia over an 18 hour period (FIG. 6). Exponential fits to the two data sets (with NONMEM) suggest that treatment with nicotine causes the analgesic action of morphine to be reduced to a significantly higher steady state level with a slower time course (T in our interaction model). To better predict the plateau effect of nicotine on morphine tolerance, we will repeat the experiment that we show in FIG. 6 over a longer time course. Specifically, we will repeat the experiments described above for FIG. 6 over a 5 day period. After the experimental surgery, the mouse will have a mini-osmotic pump implanted that will release morphine at 2 mg/kg/hr. At that time the mice will also be given a single IP injection of nicotine 1.5 mg/kg (ED₂₀ for primary hyperalgesia). The reduction in morphine analgesic effect over the 5 day time course will be measured as threshold for response to von Frey fibers and latency to response to heat. These values will be measured every 4 hours over a 5 day period.

Experiment 2b: Dose Response Relationship For Nicotine's Alteration In Morphine Tolerance.

The experiment described above in 2a will be repeated with different doses of nicotine in the concentration range that was efficacious for analgesia. We will determine whether the increase in the time course of desensitization (T) or the increase in the plateau phase independent of nicotine dose.

Experiment 2c: Effect of Nicotinic Activation change in Morphine ED₅₀ over time

Tolerance is traditionally measured as change in ED₅₀ over time. Cumulative dose response curves for morphine will be constructed for the purpose of estimating ED₅₀ values, 95% confidence intervals and equi-analgesic doses. For these studies, groups of 5 mice will be administered each morphine dose IP in 50 ul 0.9% saline across the concentration range 0-64 mg/kg (8 total injections) spaced at 30 min intervals. Analgesia will be assessed using a thermal 15 min after injection at which point we have observed maximum analgesia to occur previously. This dose vs. response curve will be repeated at 4, 8, 16 and 24 hours in the presence and absence of nicotine (1.2 mg/kg IP). The raw data will be converted to % MPE using the formula: % MPE=[(observed latency−baseline latency)/(20−baseline latency)]×100. Data will be analyzed using variable-slope sigmoidal curve fitting in NONMEM.

Expected Results and Potential Issues

There is some evidence for cross tolerance between opioids and nicotinic agonists (Zarrindast, Khoshayand et al. 1999). We were not surprised that the maximal analgesia was increased in animals treated with nicotine (FIG. 5) but we were surprised that the time course of the development of tolerance to morphine was slowed (FIG. 6). Nicotinic receptors in the nucleus acumbens play a role in both antinociception induced by a noxious stimulus induced and opioid-mediated analgesia (Schmidt, Tambeli et al. 2001). Perhaps activation of both systems simultaneously alters the process of p-opioid receptor desensitization. These experiments will further define the concentration range for nicotine that is active for opioid tolerance. We do not anticipate difficulty performing these experiments or in the data analysis as it has been done for the experiments shown in FIG. 6. These experiments would be improved if we had an assay of morphine and nicotine concentrations in our mouse model, so we could model concentration vs response relationship rather than dose vs. response relationships. Unfortunately, taking an adequate blood sample from the mouse will certainly kill it, precluding measuring the response in an individual mouse over time. We have not developed an assay for morphine or nicotine in mouse blood, but intend to pursue finding whether such an assay is feasible over the next 6 months.

Experimental Series 3: Effect of Nicotinic Activation on Inflammation

The recently described “cholinergic anti-inflammatory pathway” is a mechanism by which activation of the vagus nerve releases acetylcholine. Acetylcholine activates of α7 nicotinic receptors on macrophages, which inhibits the production of the inflammatory cytokine TNF-α, (Bernik, Friedman et al. 2002). A similar mechanism has been identified in the CNS through activation of α7 nicotinic receptors on microglia in the central nervous system (Shytle, Mori et al. 2004). It is possible that the prolonged antinociceptive action of nicotine in our human pilot study was due to peripheral anti-inflammatory effects of nicotine. In this experimental series, following surgical incision, the mice will be injected with nicotine at concentrations from 0-5 mg/kg. We will test the resulting increase in pain sensitivity at as above with response to heat and threshold for von Frey fiber applied immediately adjacent to the wound. Immediately after testing the mouse will be killed with CO₂ and a blood sample will be obtained by cardiac puncture. This procedure will also be done on 10 sham operated controls. The serum will be tested for levels of TNF-α, and the downstream cytokines, IL2 and IL6 with a fluorescently linked ELISA described in the methods section. Our methodology allows us to test a small plasma sample against multiple inflammatory mediators that could be potentially involved nicotine's action. We will test our samples against all potential targets with the hope of bringing out information about specific downstream pathways involved. Changes in levels of inflammatory mediators will be contrasted with effects on pain sensitivity. For purposes of clarity, we have presented this experimental series separately, but immediate post-mortem blood samples will be taken by cardiac puncture from a subset of animals tested in Experimental series 2 at 2 hours, 24 hours and at 5 days.

Expected Results and Potential Issues

Surgery and anesthesia initiate an inflammatory response that is important in wound healing but also can be related to increased levels of pain. We anticipate that TNF-α, IL2 and IL6 will be elevated in animals after surgery when compared to sham operated animals. There is evidence that native cholinergic activation as part of the stress response to injury acts as a break on this inflammatory pathway and that exogenous nicotine inhibits these inflammatory markers (Wang, Yu et al. 2003). As such we anticipate that animals treated with nicotine will have lower levels of TNF-α, IL2 and IL6 than animals in the placebo group. Further we expect that TNF-α level will have a negative correlation with analgesic response at 24 hours. Another potential outcome is that the inflammatory markers will be reduced by nicotine, but the level is unrelated to the animal's pain state. A third possible outcome is that there will be no measurable increase in these inflammatory mediators after our surgical protocol. This outcome would be surprising, but if so, we could use a more traditional paradigm to induce inflammation such as carageenen injection. We will learn from any of these outcomes. As inflammation is inextricably entwined with post-operative pain, we believe it is essential to understand how nicotine, in combination with morphine, might alter the body's inflammatory response to acute injury.

Experimental Series 4: Effect of Nicotinic Activation on Neuronal Plasticity

The postoperative incisional model described in the experiments above not only induces primary hyperalgesia, but secondary hyperalgesia is also induced in the area surrounding the wound during the first 24 hours (Zahn and Brennan 1999). It is possible that the pain relieving effects of nicotine in the first day after surgery are due to prevention of secondary hyperalgesia. We will study the effect of nicotine on the area of secondary hyperalgesia surrounding the experimental wound 2 hours after surgery. Baseline von Frey sensitivity will be measured for each animal before surgery. After surgery we will determine the threshold for sensitivity to pressure immediately surrounding the wound. The area of the paw that is sensitive to pressure induced by the von Frey fiber one larger than stimulates the wound will be measured in millimeters. We will determine whether nicotine significantly and dose dependently reduces the area of secondary hyperalgesia that develops after surgery. As in Experimental series 3, this series is presented as a separate section for purposes of clarity, but the effect of nicotine on the area of secondary hyperalgesia will be measured in the same animals that will be used in experimental series 1 a and b with metanicotine, DBMX-anabaseine alone and in combinations with morphine in order to not use extra experimental animals.

Expected Results and Potential Issues

We expect to be able to detect an area of secondary hyperalgesia surrounding the wound at 2 hours after surgery. We predict that that area will be significantly reduced by DBMX-anabaseine because of known effects on plasticity. If there is no effect with either subtype selective agonist, we will conduct these experiments on separate animals using nicotine. We do not anticipate difficulty with performance of this protocol.

Experimental Series 5: Timing of Nicotine Administration

To guide our clinical trials, we need to know how to optimally give nicotine relative to the surgical stimulus and the morphine administration. This will be accomplished in a series of mouse studies characterizing the nicotine-morphine response surface with differing nicotine administration paradigms. Each of these studies will be nearly identical to the original study (FIG. 5). However, the studies will look at the following dosing paradigms:
1. Animals will be randomized to receive nicotine 0.25 to 1.5 mg/kg, or placebo 2 hours prior to the hind-paw surgery, or 2 hours after the hind paw surgery (reproducing the original study). Morphine will be given as in the original study. The doses will be determined in a dose-finding portion of the interaction study. The interaction surface will be modeled and tested to identify any benefit from preoperative administration of nicotine.
2. A first group of animals will be randomized to receive nicotine or placebo via a micro-osmotic pump (Alza, Mountain View, Calif.), implanted 5 hours prior to the paw surgery, that releases nicotine at 0.25-1.5 mg/kg/24 hours (range to be decided in the dose-finding part of the interaction study). Morphine and nicotine or placebo (the test article not in the osmotic pump) will be given 2 hours after surgery. The interaction surface will be characterized at 30 minutes, 2 hours, and 4 hours after morphine administration. The study will help to document whether there is any benefit to maintaining nicotine with a constant infusion vs. a simple bolus injection.
   Expected Results and Potential Issues

Given that nicotine is not associated with “pre-emptive” analgesia, we believe that there will be no benefit from pre-operative administration of nicotine. However, we do expect to see a more stable level of analgesic response when nicotine is given by constant infusion. We do not anticipate difficulty with performance of this protocol, and the results will guide the design of future clinical trials.

Specific Aim 2: To test the hypothesis that acute post-operative pain following gynecological surgery is reduced by the perioperative administration of intra-nasal nicotine, independent of the general anesthetic.

Rational and Significance: The results of our pilot study (FIGS. 2 and 3) demonstrate with high statistical confidence that nicotine nasal spray reduces pain in women after uterine surgery under isoflurane anesthesia. Although the analgesic effect of nicotine in this small pilot study was remarkably unambiguous, it is not clear whether nicotine is acting as an analgesic itself, is potentiating the opioid analgesic, or is reversing the hyperalgesic effects of isoflurane. Volatile anesthetics such as isoflurane are known to increase pain sensitivity in the postoperative period (Dundee and Moore 1960). We have previously demonstrated in animal models that this sensitization can be reversed by nicotine (Flood, Sonner et al. 2002). Nicotine's analgesic properties in the postoperative setting could be due to the reversal of isoflurane induced hyperalgesia. If nicotine were acting specifically to reverse isoflurane induced hyperalgesia, we would expect a greater benefit in patients who had an isoflurane anesthetic than in those who received propofol. Propofol, an intravenous anesthetic, does not cause hyperalgesia. As such, we have designed a larger study that will serve to will compare the effect of nicotine on postoperative pain and PCA morphine utilization in subjects treated with two different anesthetic techniques.

Overview: This is a prospective, randomized, double blind, placebo controlled study of women scheduled to undergo hysterectomy or myomectomy at New York Presbyterian Hospital. Women were chosen because studies in animals show that volatile anesthetics enhance pain more in females than in males. All surgeries will be uterine, through a low horizontal incision, in order to best standardize surgically induced pain. The study subjects will be randomly assigned to receive one of two standardized anesthetics: an isoflurane-based anesthetic, or a propofol-based anesthetic. The subjects in both anesthetic groups will be randomly assigned by the research pharmacy to receive either nicotine nasal spray or saline placebo spray at the end of their anesthetic. The pharmacy will supply the test article in a blinded container. Both physicians and patients will be blinded to the treatment group. All patients will have morphine PCA at the conclusion of their surgery. A research coordinator, unaware of treatment group, will collect information on pain, opioid utilization, hemodynamic values, nausea, vomiting, and pruritis during over the first 48 hours of the postoperative period. Our primary outcome variable will be the VAS score (1-10) in the first hour after surgery in patients receiving nicotine vs. patients receiving placebo, stratified by anesthetic regimen. The secondary outcome will be morphine utilization over the first hour in patients receiving nicotine vs. patients receiving placebo, again stratified by anesthetic regimen.

Protocol: This study has been approved by the institutional review board at New York Presbyterian Hospital. All subjects will provide written informed consent. The subjects will be randomly assigned to receive nicotine nasal spray (3 mg) or saline placebo after being anesthetized with one of two different standard general anesthetic techniques that is also randomly determined. As such there will be four study groups:

Group 1: isoflurane anesthesia-nicotine nasal spray

Group 2: isoflurane anesthesia-placebo nasal spray

Group 3: propofol anesthesia-nicotine nasal spray

Group 4: propofol anesthesia-placebo nasal spray

The clinical anesthesiologist and patient will be familiarized with the study protocols, the use of the VAS pain score, and operation of the PCA device prior to the surgery. After placing an intravenous catheter and standard anesthetic monitors, the patient will be pre-oxygenated. Fentanyl will be administered with a bolus dose of 1-2 μg/kg and a continuous infusion of 1-2 μg/kg/hr will be begun. Anesthesia will be induced with propofol 2 mg/kg and intubation facilitated with succinylcholine 1-2 mg/kg. Following intubation, anesthesia will be maintained with the addition of either inhaled isoflurane or intravenous propofol (depending on group assigned) titrated by the anesthesiologist to clinical effect. Muscle relaxation will be maintained with vecuronium as needed. Equivalent depth of anesthesia will be verified by maintaining BIS at a value of approximately 50 (Sennholz 2000). Hypotension will be treated with intravenous fluid, ephedrine, or phenylephrine as deemed necessary by the anesthesiologist. Hypertension will be treated with hydralazine, metoprolol, or labatelol, in doses determined by the anesthesiologist. If other hemodynamic or anesthetic drugs are deemed necessary by the anesthesiologist, the patient will be removed from the protocol.

Approximately 5 minutes before anticipated completion of the surgery the anesthetic will be titrated to off. Residual neuromuscular blockade will be reversed with neostigmine 3 mg and glycopyrrolate 0.6 mg. The study drug (nicotine 3 mg or saline) will be administered by intranasal spray, with half of the volume administered to each nostril. The patient will be extubated by the anesthesiologist when she meets normal criteria (as determined by the anesthesiologist).

Within five minutes after extubation, the patient will be asked for a visual analog pain score (VAS) score by the study coordinator. The VAS score is a number from 0 to 10 where 0 is no pain and 10 is the worst pain imaginable. PCA morphine will be immediately available at the conclusion of surgery as follows (1 mg bolus dose, a lock-out of 6 minutes and a maximal 1 hour dose of 10 mg). A rescue dose of 3 mg morphine is available to be administered by the nurse through the PCA every 5 minutes for a maximum of 12 mg in 4 hours if the patient is not over sedated (patient responsive to voice), is hemodynamically stable, and has a respiratory rate greater than 12 breathes per minute. If pain is inadequately treated there will be an option to increase the patient demand dose to 1.5 mg morphine and the 1 hour maximum to 15 mg. This is a typical PCA protocol used at our institution for this surgery.

Patients will have standard monitors in the post anesthetic care unit except that the patient's pain intensity and hemodynamic values will be monitored at least every 5 minutes in the first hour, then at 2, 3, 4, 6, 12, 24, 36, and 48 hours postoperatively by the study coordinator. PCA utilization will be determined from the amount of morphine administered by the PCA machine and the nursing records from the PACU. Any episodes of nausea, vomiting or pruritis will be noted by the study coordinator and treated as per recovery room routine. Patients will be identified only by a sequential numbering system and the data will be stored on a computer and in a locked cabinet in the Principle Investigator's office. The resulting data will be analyzed with ANOVA for repeated measures using NONMEM. All data will be included and analyzed according to the intention to treat.

Data Analysis: The data will be analyzed using a population approach, implemented in NONMEM on the intent-to-treat study population. The primary outcome variable: the influence of nicotine on VAS score, will be modeled on the assumption that the interindividual variability in VAS score from observation to observation is consistent, reproducing the standard assumption in repeated measures analysis of variance. This assumption will, however, be specifically tested by examining the change in objective function if the interindividual variance parameter is allowed to differ at specific time points (typically very early or very late in the study). Interindividual and intraindividual variability will be modeled using simple additive models. Statistical significance will be inferred from the improvement in −2 log likelihood with the addition of each study covariate (i.e., nicotine effect vs, no nicotine effect, drug and nicotine effect vs. nicotine effect only). As differences in −2LL follow a χ2 distribution, differences in −2LL of 3.8 or higher (χ²<0.05,1) with the addition of a single model parameter will be considered statistically significant.

Using similar population methods we will also analyze a several important secondary variables, including:
1. the relationship between nicotine and morphine usage,
2. and the influence of anesthetic technique on the relationship between nicotine and morphine usage,
3. the relationship between nicotine and nausea and vomiting,
4. the influence of anesthetic technique on the relationship between nicotine and nausea and vomiting,
5. the relationship between nicotine and blood pressure and heart rate, and
6. the influence of anesthetic technique on the relationship between nicotine and blood pressure and heart rate.

Power analysis: We would consider finding a difference in VAS score of 2 points to be clinically significant. In our preliminary data set, the average VAS score for patients receiving nicotine was approximately 4.5 in the first hour, while the average VAS score for the patients receiving placebo was 6.5. The standard deviation in both groups was 2.2. With a level of significance of 0.05, and a power of 0.9, the number of subjects per group should be 20. However, this does not capture the repeated measures aspect of the study design, which increases the study power. The power analysis was therefore refined using a bootstrap analysis of the initial patient data. We performed 100 simulations of clinical trial results. Each simulation was performed by drawing at random 20 nicotine treated patients and 20 placebo treated patients from the patients in the preliminary data. The resulting populations were analyzed with NONMEM. Of the 100 simulations, the effect of nicotine was significant at p<0.05 in 96 simulations, and at p<0.01 in 88 simulations. Thus, based on the bootstrap analysis, 20 subjects per group offers a highly sensitive study design, with a (conservatively estimated)>80% probability of identifying a 2 point improvement in VAS score at p<0.01.

The bootstrap analysis confirms that 20 subjects per group should suffice to identify the effect of nicotine, but it does not directly speak to the proposed study design. In the study proposed for Specific Aim 2, half of the subjects will receive isoflurane (as in the pilot study) and half the patients will receive propofol (for which we have no data). We performed a fully parametric power analysis of this proposed study using “computer-assisted trial design”. Patients receiving isoflurane, with nicotine or placebo, were presumed to have the time course, interindividual variances, and residual intraindividual variances that were estimated by NONMEM for the pilot data. As a first step, we verified that parametric power analysis of a study with 20 subjects per group, all receiving isoflurane, produced similar results to the gold standard bootstrap approach. To extend the model to patients receiving propofol, we postulated that propofol caused a 75% reduction in nicotine analgesic activity. This is a worst case scenario—assuming the drug effect of interest nearly disappears in half of the study population. Using NONMEM, we simulated 100 clinical trials. In each simulated trial there were 80 patients, stratified into 40 patients receiving isoflurane and 40 patients receiving propofol. Within each group of 40 simulated patients, 20 simulated patients received placebo and 20 simulated patients received nicotine. In the 100 simulations, 93 identified the nicotine effect at p<0.05, and 81 identified the nicotine effect at p<0.01. Similarly, 87 simulations identified the effect of anesthetic at p<0.05, and 66 simulations identified the effect of anesthetic at p<0.01. Thus, with conservative assumptions, the proposed study with 80 subjects has at least an 80% power to identify the primary outcome variable (nicotine analgesic effect) at p<0.05, and also has at least an 80% power to identify the secondary outcome variable (influence of anesthetic effect) at p<0.05.

Expected Results and Potential Issues

We expect to be able to identify 1) the influence of anesthetic choice on the duration of nicotine analgesic effect, and 2) the duration of nicotine analgesic effect. There are, however, several potential pitfalls in the experimental design:
1. The study does not incorporate a dose vs. response assessments. We agree on the importance of understanding both dose vs. response and concentration vs. response assessments. However, this is a translational research program. The animal studies in this grant will include fundamental pharmacological characterization, including dose vs. response relationships, the nature and extent of synergy, timing of drug administration (given before vs. concurrent with morphine, and by single bolus vs. continuous infusion), and persistence of effect. When these results are known in animals, we will then translate these findings into a full exploration of the analgesic response surface (Minto, Schnider et al. 2000) for nicotine and morphine in a state-of-the-art volunteer trial using target controlled drug administration, the “cross-cross” design in volunteers and frequent arterial blood samples for pharmacokinetic analysis (Short, Ho et al. 2002).
2. The study does not measure morphine or nicotine concentrations. Although we do not believe that nicotine is exerting its antinociceptive effects by inhibiting morphine metabolism, it is possible as both undergo glucuronication in the liver. The volunteer interaction study, described above as the next steps in this translational program, will incorporate very detailed pharmacokinetic evaluations of nicotine and morphine based on frequent arterial blood samples. As we consider the possibility of pharmacokinetic interaction remote, we would prefer to not subject patients to extensive blood sampling for PK analysis.
3. Another limitation of this study is that inflammatory markers are not being measured. The reason this was not proposed is that this study has already started, and we did not envision measuring the inflammatory response when the study was designed a year ago. However, we intend to measure inflammatory markers in all future studies of nicotine analgesia until we are certain whether or not nicotine modulates the inflammatory response.

Specific Aim 3: To test the hypothesis that intra-nasal administration of nicotine reduces post-operative pain after third molar extraction in men and women patients stratified by smoking history. This model will also test whether nicotine has anti-inflammatory effects that contribute to post-operative analgesia.

Rational and Significance: This study will examine the influence of nicotine on analgesia in men and women using a third-molar extraction surgical model. There are several advantages of the third molar extraction model to further our studies on nicotinic analgesia.
1. This surgery is equally performed in men and women providing an ideal population do determine whether nicotine is equally efficacious in both genders. We have found in our mouse model that the action of nicotine to reverse isoflurane hyperalgesia depends on estrogen (Flood and Daniel 2003). In a study of male and female human volunteers however, the men had more analgesic effect of nicotine then the women (Jamner, Girdler et al. 1998; Guthrie, Zubieta et al. 1999).
2. Third molar extraction is a moderately painful procedure typically performed under local anesthesia with or without sedation. Patients who require extraction of all four third molars are typically treated at our institution in 2 visits with 2 teeth extracted on each visit. As such these patients can be studied with a cross over design, with each patient acting as his/her own control. The simulations described in our power analysis suggest that this will be an unusually effective strategy for analgesic trials, potentially an interesting outcome in and of itself.
3. The pain continues for several days after surgery.

As such we will be able to determine whether nicotine has an action that outlasts its apparent plasma half life and if so, for how long.
4. Pain after this procedure is at least partially inflammatory in nature and it is commonly treated with a combination of acetaminophen and narcotic or non-steroidal anti-inflammatory drug combined with narcotic (Swift, Garry et al. 1993; Roszkowski, Swift et al. 1997). Since there is a prominent inflammatory component to the pain resulting from this surgery, the potential role of nicotine as an anti-inflammatory agent can be addressed both by assessing the clinical efficacy of nicotine in this model and through biochemical analysis of inflammatory mediators in plasma that are thought to play a role in nicotine's anti-inflammatory actions. There is evidence from animal models that activation of α7 type nicotinic receptors on macrophages could have an anti-inflammatory action (Wang, Yu et al. 2003). Decreased inflammation could account for the analgesic effect of nicotine and we would expect such an action to be demonstrable in this paradigm.
5. Third molar extraction is most commonly done in young healthy patients. Because of the ability to enroll healthy smokers as well as healthy non-smokers, we will be able to assess nicotine's effectiveness as an analgesic in these populations for comparison. Immediately after surgery, the smoker has issues of nicotine withdrawal, in addition to pain. As such, nicotine treatment might be particularly beneficial in this group. On the other hand, chronic exposure to nicotine by smoking results in a down regulation of nicotinic receptor number and tolerance to the effects of nicotine.

Smokers might have less effect from nicotine than non-smokers. However, experimentally induced pain in a volunteer population was equally relieved by nicotine in male smokers and non-smokers (Jamner, Girdler et al. 1998).

Protocol: This study has been approved by the Institutional Review Board at New York Presbyterian Hospital. The first subject has been enrolled. All patients will give written informed consent. The study will be conducted in the outpatient oral surgery clinic supervised by Dr. Rosenberg. Patients will be informed about the study by their dental surgeon and recruited if they are interested in participating. Only patients who require extraction of all 4 third molars will be eligible for enrollment. Molars will be extracted by the oral surgeons in 2 sessions of 2 teeth each session as is the usual practice. In one session the patient will receive nicotine nasal spray and in the other, placebo. Subjects will be randomly allocated to receive either nicotine nasal spray (Nicotrol NS 3 mg) or an equal volume of sterile saline (prepared by a random allocation table by the research pharmacy) at the conclusion of their surgery while the local anesthetic is still active.

Patient's enrollment will be stratified by gender and smoking status (i.e., male smokers, male non-smokers, female smokers and female non-smokers). Patients who smoke at least 1 pack of cigarettes/week will be considered smokers and those who have not smoked more than 1 pack of cigarettes or the tobacco equivalent in their lives will be considered non-smokers. Smokers will be identified by self report. All patients will be greater than 18 and less than 60 years of age. Exclusion criteria include chronic pain syndrome, current opioid use, uncontrolled hypertension or cardiac disease.

Patients will be sedated with midazolam, 0-4 mg iv, prior to the beginning of the procedure. Patients will then have a block placed by the surgeon per their routine for third molar extractions. The block will be performed with lidocaine with epinephrine 1/10,0000, 2-3 cc to upper and lower jaw. The local anesthetic block usually lasts approximately one and one half hours. The surgery takes one half to one hour.

The patients will spend at least 2 hours in recovery during which time they will be asked their visual analog pain score (0-10, where 0 is no pain and 10 is the worst imaginable) every 5 minutes for the first hour and every 15 during the second hour. Blood pressure, heart rate, respiratory rate, incidence of vomiting and a VAS score for nausea will be recorded at these same time intervals. The time to first request for pain medicine will also be recorded. All patients will be prescribed hydrocodone/acetaminophen (Vicodin) 1-2 tablets every 4 hours by their surgeon. Patients will be contacted by the study coordinator each morning on post operative days 1, 2, 3, 4, and 5 to inquire about VAS score, the number of Vicodin tablets taken during the previous day and any nausea, vomiting or pruritis.

Blood will be taken prior to surgery, 2 hours after surgery, 24 hours after surgery for assay of TNF-α, IL2, and IL6. The 24 hour sample will be taken at the subject's home.

Data Analysis: The data analysis will be very similar to that described in Specific Aim 2. NONMEM will be used to perform the population analysis. The primary outcome variable will be the influence of nicotine on VAS score. Secondary outcome variables will be the influence of gender and smoking on the relationship between nicotine and analgesia. We will also analyze as secondary outcome variables
1. the relationship between nicotine and Vicodin usage,
2. and the influence of gender and smoking on the relationship between nicotine and Vicodin usage,
3. the relationship between nicotine and first use of Vicodin,
4. and the influence of gender and smoking on the relationship between nicotine and first Vicodin usage,
5. the relationship between nicotine and nausea and vomiting,
6. the influence of gender and smoking on the relationship between nicotine and nausea and vomiting,
7. the influence of nicotine on TNF-α, IL2, and IL6,
8. the influence of nicotine on subject drop-out.

The need for two sessions significantly increases the risk of subject dropping out. We will analyze drop-outs for a relationship to nicotine, as dropping out of the study is potential a source of bias for or against the primary endpoint (i.e., subjects with high levels of pain in the first session may be less likely to have the remaining wisdom teeth removed). In general, the population approach is thought to help exclude bias from non-random data censorship. However, if there is a relationship between nicotine and drop-out, we may revert from an intent-to-treat analysis to a per-protocol analysis to remove the possible bias of non-random data censorship.

Power analysis: As described for Specific Aim 2, complex study designs are best assessed using the principles of simulation and computer-assisted trial design. We postulate that the analgesic effect of nicotine in men is just 25% of the analgesic effect of nicotine in women, and that the effect in smokers is just 25% of the effect in non-smokers. In both cases, these are fairly worst-case assumptions: the effect of interest is greatly reduced in smokers, and in men, and nearly disappears in smoking men. However, unlike the power analysis for Specific Aim 2, in this case each subject has two sessions, one with and one without the primary study drug (nicotine). The simulations also postulated 20% interoccasion variability, reflecting the study subject's need for two separate visits. As before, we simulated 100 clinical trials of 80 subjects each.

Even with these multiple assumptions, every one of the 100 simulated studies demonstrated the nicotine effect, the postulated smoking effect, and the postulated gender effect at p<0.01. This speaks for the potential power of this cross-over study design. The study may, in fact, be overpowered for the primary endpoints. However, it is very feasible as currently powered, and the use of a well powered design will facilitate exploration of the secondary endpoints. Additionally, the power analysis did not include drop-outs, which will potentially reduce the power of the study.

Expected Results and Potential Issues

We expect to characterize the relationship between nicotine and morphine in a second surgical paradigm: third molar extraction, as well as to determine the influence of smoking and gender on the interaction. We also expect to determine the influence of co-administration of nicotine on markers of inflammatory response, specifically TNF-α, IL2, and IL6. The study shares the same pitfalls as described for Specific Aim 2: lack of dose vs. response information, and lack of pharmacokinetic analysis. As mentioned above, we intend to address the potential for pharmacokinetic interaction in a volunteer study that is still being designed. A second potential pitfall is the lack of preliminary data from the investigator's laboratory showing the influence of nicotine on the inflammatory response, and data from the investigator demonstrating the ability to assay for markers of inflammatory response. The role of inflammation in pain response is so unambiguous, and the anti-inflammatory effects of nicotine are sufficiently documented in prior studies (Wang, Yu et al. 2003) that we do not believe preliminary data are necessary to justify measuring inflammatory markers in an post-surgical analgesia trial. We do not intend to design and validate our own assay for the markers of interest. Instead, we will be using “off the shelf” technology for the determinations, which we will validate per the vendor's recommendations. The vendor has validated their assay against other widely accepted assays for murine and human inflammatory markers, and this information is publicly available.

Methods

1. Surgical Procedure—Post Operative Pain Model (Aim 1):

Mice will be anesthetized with 1.5% to 2.5% isoflurane in oxygen until there is no response to a paw and tail pinch. Alcohol 70% is swabbed on the foot before the surgery began as an antiseptic measure. Next a 5 mm longitudinal incision is made with a no. 15 blade through the skin and fascia of the plantar foot. The incision is started 2 mm from the proximal edge of the heel and extended toward the toes. The underlying muscle is elevated with forceps, leaving the muscle origin and insertion intact. Finally the skin is apposed using a single polysorb suture, and the wound covered with an antibiotic ointment. The mice are then allowed a 2 hour recovery period before behavioral testing begins.

2. Implantation of Mini-Osmotic Pump (Opioid Tolerance Experiments):

While under isoflurane anesthesia, after the hind paw surgery above, a small area behind the neck is shaved and a small incision made in the skin between the scapulae. Using a modified probe a pocket is formed by separating the subcutaneous connective tissue. An ALZET Mini-Osmotic Pump is inserted into the pocket. The skin incision is then closed using 3 6.0 chromic sutures.

The mice will be injected with either nicotinic agonist or saline in equal volume and then will be allowed to recover from anesthesia for two hours before pain testing began. Once the recuperation period is over pain scores will be measured using hind paw withdrawal latency and von Frey filament assays during continuing infusion of morphine.

3. Behavioral Testing

a. Withdrawal Latency to Heat:

We will measure the latency to response to an infrared heat stimulus in the injured hind paw in up to five unrestrained mice (per study) housed individually in clear plastic chambers. The chambers rest on a clear glass plate that is warmed to minimize body heat loss. To diminish exploratory activity, the mice will be acclimated to this environment for at least 30 minutes before commencing the study. After acclimation, a movable source of radiant heat will be applied from a lamp through an aperture under the glass plate to the hind paw of the resting mouse. The testing stimulus used will be 15% of maximal intensity that caused an average increase to. 42° C. on movement. An investigator blinded to drug dose will measure the time from the onset of the stimulus to the time the mouse moved the hind limb.

b. von Frey:

The mice will be placed on an elevated mesh floor and enclosed in clear plastic chambers. To reduce exploratory activity, the mice will be allowed to acclimate to this environment for approximately 30 minutes before testing. von Frey filaments will be pushed up through the mesh flooring and against each mouse's injured hind paw, immediately proximal to the incision. Each von Frey filament is calibrated to a specific value of grams of force that result in bending of the fiber. A response to a filament will be identified as the withdrawal of the paw when pressure is applied for 1 second. The von Frey filaments will be applied in order of increasing pressure until a paw withdrawal takes place. The filament's pressure value and that of the previous filament are averaged to provide the value of force intermediate between response and non-response. These tests are performed 5 times on each paw to ensure accuracy. The value in grams of bending force values are converted into milliNewton units (mN) by multiplying by a factor of 9.8. The maximum fiber tested will be 10 gms (fiber #10 in the Harvard Apparatus fiber set). Animals not responding to 10 gms of force will be assigned a value of 15 gms (intermittent between 10 and 15 g fibers). The investigator performing the assessment will be blinded to the drug received.

4. Inflammatory Markers:

The inflammatory markers that will be measured (TNF-α, IL2 and IL6) will be measured with the same methodology for both human and mice studies. This method has the benefit of using small samples to measure cytokine concentrations within a large dynamic range so that small samples from mice can be used. A plasma sample is obtained from whole blood by centrifugation. The entire assay takes place in a single 96 well plate. The sample (200 μl) is incubated with antibody coupled beads for 30 minutes. After a wash, the sample is incubated with biotinylated detection antibody for 30 minutes. After a second wash, the sample is incubated with streptavidin-PD for 10 minutes and the resulting fluorescence can be read on the Bioplex system. This assay has the sensitivity to detect cytokines at concentrations less than 10 pg/ml at greater than 2 SD above baseline. Inter and intra assay variability is less than 10% at concentrations from 1-32,0000 pg/ml. Cross reactivity between related cytokines is negligible. Additional description and validation can be found on the Biorad website.

5. Secondary Hyperalgesia (Sensitization):

For experiments in Aim 1, Experimental Series 4, we will measure the area of secondary hyperalgesia that is induced by the surgical incision. Before surgery, the threshold for withdrawal will be measured as above. Surgery will be performed as described above and the area in millimeters surrounding the incision that is responsive to the test fiber will be measured. A series of pen dots at 1 millimeter intervals will be made on the animal's foot in a radial array from the incision while the animal is under anesthesia. At two hours after surgery, the animals will be tested for threshold for primary hyperalgesia by probing immediately adjacent to the wound. Testing for secondary hyperalgesia will be done with the next larger von Frey fiber that did could illicit primary hyperalgesia. The area of secondary hyperalgesia will be calculated as the area of a rectangle with a width twice the distance from the wound in one direction and a length the length of the wound plus twice the distance.

6. Human subjects:

The two clinical studies are currently approved by our Institutional Review Board and enrollment for both studies has started.

The subject's privacy will be protected by having all data de-identified and all records will be kept under lock and key in the Principle Investigator's office. There is some risk of increased blood pressure or heart rate after treatment with nicotine. The average age for the gynecological patients was 45 in our pilot study and will likely be the same in the study for AIM 2. The subjects studied in AIM 3 will likely be younger as third molars typically erupt in adolescence. All subjects will be American Society of Anesthesiologist Classification 1 or 2 (with no systemic disease or with well controlled systemic disease) and would be at low risk of severe hemodynamic consequences of a single dose of nicotine. However, all subjects will be monitored for at least 2 hours in a post-anesthesia recovery setting to ensure hemodynamic stability. No hemodynamic consequences have been identified in our pilot study.

Nicotine is an addictive drug. However, a single dose of nicotine will be given under either general, local anesthesia or conscious sedation. Many of the subjects will be non-smokers and it is unlikely that the effects of nicotine will be generalized to smoking. Although nicotine is addictive, opioid narcotics are also addictive and there is the potential to decrease the amount and side effects of these drugs as well as the potential for decreased opioid tolerance. We are unaware of any data suggesting that a single dose of nicotine leads to nicotine dependence.

The subjects will be referred by the surgeons and contacted prior to the day of surgery to determine whether they are willing to discuss a research project. If they agree, the project will be discussed by telephone and written informed consent will be obtained on the day of surgery.

7. Sources of Material:

5 ml of blood will be drawn from subjects in study 2 (Specific Aim 3) after each surgery. Concentrations of inflammatory markers will be measured and compared between the surgery with nicotine and that without. Samples will be marked with study code only and will have no identifying personal information. After assay for inflammatory markers all samples will be discarded.

8. Inclusion of Women and Minorities:

The majority of subjects to be studied are women. This is because preliminary animal studies suggested that this treatment might be particularly useful in women. A total of 80 patients having hysterectomy or myomectomy will be studied. Obviously these will all be women. Additionally, 40 men and 40 women having their third molars removed will be studied in two sessions. This stratification will allow us to detect treatment differences between men and women.

New York Presbyterian Hospital has a patient population that is approximately 40% Hispanic and 20% African American and 40% other ethnicities. We have all of our consent forms translated into Spanish and do not anticipate difficulty enrolling minority subjects in proportion to the patient population.

9. Inclusion of Children:

We plan to include children between the ages of 18-21 in both of the planned studies. This age group will likely not be heavily represented in the study of women having hysterectomy and myomectomy, but will include many subjects having third molar removal. We are reluctant to enroll younger children in these preliminary studies until more is known about the benefits to adults. This is because the effects of nicotine during development are not known.

10. Vertebrate animals:

a. Mice will be used in an animal model of postoperative pain that has been approved by the IACUC at Columbia University. We will use female 129J mice from Jackson laboratories at 6-10 weeks of age for most experiments for consistency with previous work. We anticipate using a total of 205 animals.
b. Pain is a complex behavior and post-operative pain is specific in that it involves particular responses to tissue injury that are integrated in the CNS. As such it would not be possible to study such a complex behavior in anything other than an animal model.
For experimental series 1, the intra-individual variability in our previous experiments as calculated with NONMEM would suggest that groups of 5 animals per dose is sufficient to calculate a “typical response”.
We will test 5 doses for each concentration response relationship. Since morphine will be used as the prototypical opioid agonist in all cases, this relationship will not need to be repeated. A concentration response for each drug in the presence of the ED₂₀ dose of the other drug will be calculated.
This is the most efficient method for creating a response surface (Short, Ho et al. 2002). Since the 0 concentration need not be retested for the combinations, 13 concentrations will be tested for each experiment for a total of 65 mice in 2 experiments for a total of 130 mice.
Experimental Series 2 will test the effect of 2 subtype selective nicotinic agonists on the time course of development and extent of tolerance to morphine. Because of the time course of development of tolerance will be tested in 10 animals per condition or a total of 20 animals because of greater variability than in other experiments above (see FIG. 5). The change in ED₅₀ will be tested with 5 doses of morphine, 5 animals each with each of 2 nicotinic agonists (50 mice). To determine the change in ED₅₀ with respect to dose these experiments will be conducted at 4 additional concentrations of nicotine for 40 additional mice. Total for this series is 110.
Experimental series 3 will test the relationship between the modulation of inflammatory mediators and pain. This experiment will be conducted concurrently with experimental series 1 except for additional experiments with nicotine if the results with metanicotine and DMBX-anabaseine are negative (65 mice).
Experimental series 4 will be conducted on the mice used in experimental series 2 and as such no additional animals will be used.
3. The animals are under the continuous care of a full time veterinarian. They are provided with ad lib. food and water and are kept on a 12 hour light dark cycle.
4. The protocol is designed to study postoperative pain. As such it is unavoidable that we create tissue injury in the form of an incision in the mouse's paw. The surgery is conducted under isoflurane anesthesia after confirmation of adequate anesthesia with lack of movement in response to a paw and tail pinch. The animal receives analgesic medication prior to emergence from anesthesia. The response to stimulation with heat and von Frey fibers is necessarily conducted during activity of the analgesic and the animal is then killed.
5. Euthanasia is done with an overdose of CO₂. This method is chosen because of its efficacy and ease. Also in some experiments a cardiac puncture is required and this is still possible after death with CO₂. This method of euthanasia is consistent with the recommendations of the Panel on Euthanasia of the American Veterinary Medical Association.

### REFERENCES

Aceto, M. D., R. S. Bagley, et al. (1986). “The spinal cord as a major site for the antinociceptive action of nicotine in the rat.” Neuropharmacology 25(9): 1031-6.
Alexander, R., H. E. El-Moalem, et al. (2002). “Comparison of the morphine-sparing effects of diclofenac sodium and ketorolac tromethamine after major orthopedic surgery.” J Clin Anesth 14(3): 187-92.
Bencherif, M., M. E. Lovette, et al. (1996). “RJR-2403: a nicotinic agonist with CNS selectivity I. In vitro characterization.” J Pharmacol Exp Ther 279(3): 1413-21.
Benowitz, N. L., S. Zevin, et al. (1997). “Sources of variability in nicotine and cotinine levels with use of nicotine nasal spray, transdermal nicotine, and cigarette smoking.” Br J Clin Pharmacol 43(3): 259-67.
Bernik, T. R., S. G. Friedman, et al. (2002). “Pharmacological stimulation of the cholinergic antiinflammatory pathway.” J Exp Med 195(6): 781-8.
Bitner, R. S., A. L. Nikkel, et al. (1998). “Role of the nucleus raphe magnus in antinociception produced by ABT-594: immediate early gene responses possibly linked to neuronal nicotinic acetylcholine receptors on serotonergic neurons.” J Neurosci 18(14): 5426-32.
Bitner, R. S., A. L. Nikkel, et al. (2000). “Reduced nicotinic receptor-mediated antinociception following in vivo antisense knock-down in rat.” Brain Res 871(1): 66-74.
Bowdle, T. A. (2004). “Nocturnal arterial oxygen desaturation and episodic airway obstruction after ambulatory surgery.” Anesth Analg 99(1): 70-6.
Brennan, T. J. (2002). “Frontiers in translational research: the etiology of incisional and postoperative pain.” Anesthesiology 97(3): 535-7.
Brennan, T. J., E. P. Vandermeulen, et al. (1996). “Characterization of a rat model of incisional pain.” Pain 64(3): 493-501.
Caggiula, A. R., L. H. Epstein, et al. (1995). “Different methods of assessing nicotine-induced antinociception may engage different neural mechanisms.” Psychopharmacology (Berl) 122(3): 301-6.
Christensen, M. K. and D. F. Smith (1990). “Antinociceptive effects of the stereoisomers of nicotine given intrathecally in spinal rats.” J Neural Transm Gen Sect 80(3): 189-94.
Colquhoun, L. M. and J. W. Patrick (1997). “Pharmacology of neuronal nicotinic acetylcholine receptor subtypes.” Adv Pharmacol 39: 191-220.
Cordero-Erausquin, M. and J. P. Changeux (2001). “Tonic nicotinic modulation of serotoninergic transmission in the spinal cord.” Proc Natl Acad Sci USA 98(5): 2803-7.
Creekmore, F. M., R. A. Lugo, et al. (2004). “Postoperative opiate analgesia requirements of smokers and nonsmokers.” Ann Pharmacother 38(6): 949-53.
Damaj, M. I., M. Fei-Yin, et al. (1998). “Antinociceptive responses to nicotinic acetylcholine receptor ligands after systemic and intrathecal administration in mice.” J Pharmacol Exp Ther 284(3): 1058-65.

Damaj, M. I., W. Glassco, et al. (1999). “Antinociceptive and pharmacological effects of metanicotine, a selective nicotinic agonist.” J Pharmacol Exp Ther 291(1): 390-8.
Damaj, M. I., E. M. Meyer, et al. (2000). “The antinociceptive effects of alpha7 nicotinic agonists in an acute pain model.” Neuropharmacology 39(13): 2785-91.
Dundee, J. and J. Moore (1960). “Alterations in Response to Somatic Pain Associated with Anesthesia IV: The Effect of Sub-Anaesthetic Concentrations of Inhalation Agents.” British Journal of Anaesthesia 32: 453-9.
Dundee, J. W. and J. Moore (1960). “Alterations in response to somatic pain associated with anaesthesia. IV. The effect of subanaesthetic concentrations of inhalation agents.” Br J Anaesth 32: 453-9.
Fertig, J. B., 0. F. Pomerleau, et al. (1986). “Nicotine-produced antinociception in minimally deprived smokers and ex-smokers.” Addict Behav 11(3): 239-48.
Fishbein, L., P. O'Brien, et al. (2000). “Pharmacokinetics and pharmacodynamic effects of nicotine nasal spray devices on cardiovascular and pulmonary function.” J Investig Med 48(6): 435-40.
Flood, P. and D. Daniel (2003). “Pronociceptive actions of isoflurane: A protective role for estrogen.” Anesthesiology 99: 1-4.
Flood, P. and D. Daniel (2004). “Intranasal nicotine for postoperative pain treatment.” Anesthesiology In Press. Flood, P., J. Sonner, et al. (2002). “Isoflurane Hyperalgesia is Modulated by Nicotinic Inhibition.” Anesthesiology 97: 192-8.
Foral, P. A., A. F. Wilson, et al. (2002). “Gastrointestinal bleeds associated with rofecoxib.” Pharmacotherapy 22(3): 384-6.
Forrest, W. H., Jr. and J. W. Bellville (1964). “The Effect of Sleep Plus Morphine on the Respiratory Response to Carbon Dioxide.” Anesthesiology 25: 137-41.
Fu, Y., S. G. Matta, et al. (1998). “Nicotine-induced norepinephrine release in the rat amygdala and hippocampus is mediated through brainstem nicotinic cholinergic receptors.” J Pharmacol Exp Ther 284(3): 1188-96.
Garraway, S. M. and S. Hochman (2001). “Modulatory actions of serotonin, norepinephrine, dopamine, and acetylcholine in spinal cord deep dorsal horn neurons.” J Neurophysiol 86(5): 2183-94.
Gillberg, P. G., P. Hartvig, et al. (1990). “Behavioral effects after intrathecal administration of cholinergic receptor agonists in the rat.” Psychopharmacology 100(4): 464-9.
Grupe, A., S. Germer, et al. (2001). “In silico mapping of complex disease-related traits in mice.” Science 292(5523): 1915-8.
Guthrie, S. K., J. K. Zubieta, et al. (1999). “Arterial/venous plasma nicotine concentrations following nicotine nasal spray.” Eur J Clin Pharmacol 55(9): 639-43.
Haxhiu, M. A., J. Mitra, et al. (1985). “Influence of central chemoreceptor afferent inputs on respiratory muscle activity.” Am J Physiol 249(2 Pt 2): R266-73.
Ho, S. T., J. J. Wang, et al. (1999). “Surgical pain attenuates acute morphine tolerance in rats.” Br J Anaesth 82(1): 112-6.
Hunt, T. E., W. H. Wu, et al. (1998). “The effects of serotonin biosynthesis inhibition on nicotine and nifedipine-induced analgesia in rats.” Anesth Analg 87(5): 1109-12.
Inturrisi, C. E. (2002). “Clinical pharmacology of opioids for pain.” Clin J Pain 18(4 Suppl): S3-13.
Iwamoto, E. T. (1991). “Characterization of the antinociception induced by nicotine in the pedunculopontine tegmental nucleus and the nucleus raphe magnus.” J Pharmacol Exp Ther 257(1): 120-33.
Iwamoto, E. T. and L. Marion (1993). “Adrenergic, serotonergic and cholinergic components of nicotinic antinociception in rats.” J Pharmacol Exp Ther 265(2): 777-89.
Jain, K. K. (2004). “Modulators of nicotinic acetylcholine receptors as analgesics.” Curr Opin Investig Drugs 5(1): 76-81.
Jamner, L. D., S. S. Girdler, et al. (1998). “Pain inhibition, nicotine, and gender.” Exp Clin Psychopharmacol 6(1): 96-106.
Jasinski, D. R. (1997). “Tolerance and dependence to opiates.” Acta Anaesthesiol Scand 41(1 Pt 2): 184-6.
Kanarek, R. B. and C. Carrington (2004). “Sucrose consumption enhances the analgesic effects of cigarette smoking in male and female smokers.” Psychopharmacology (Berl) 173(1-2): 57-63.
Khan, I. M., H. Buerkle, et al. (1998). “Nociceptive and antinociceptive responses to intrathecally administered nicotinic agonists.” Neuropharmacology 37(12): 1515-25.
Khan, I. M., M. Marsala, et al. (1996). “Intrathecal nicotinic agonist-elicited release of excitatory amino acids as measured by in vivo spinal microdialysis in rats.” J Pharmacol Exp Ther 278(1): 97-106.
Khan, I. M., P. Taylor, et al. (1994). “Stimulatory pathways and sites of action of intrathecally administered nicotinic agents.” J Pharmacol Exp Ther 271(3): 1550-7.
Kulak, J. M., J. M. McIntosh, et al. (2001). “Nicotine-evoked transmitter release from synaptosomes: functional association of specific presynaptic acetylcholine receptors and voltage-gated calcium channels.” J Neurochem 77(6): 1581-9.
Kunze, U., R. Schoberberger, et al. (1998). “Alternative nicotine delivery systems (ANDS)—public health-aspects.” Wien Klin Wochenschr 110(23): 811-6.
Lavelle, C., C. Birek, et al. (2003). “Are nicotine replacement strategies to facilitate smoking cessation safe?” J Can Dent Assoc 69(9): 592-7.
Li, P. and M. Zhuo (2001). “Cholinergic, noradrenergic, and serotonergic inhibition of fast synaptic transmission in spinal lumbar dorsal horn of rat.” Brain Res Bull 54(6): 639-47.
Li, X. and J. C. Eisenach (2002). “Nicotinic acetylcholine receptor regulation of spinal norepinephrine release.” Anesthesiology 96(6): 1450-6.
Li, X., D. G. Rainnie, et al. (1998). “Presynaptic nicotinic receptors facilitate monoaminergic transmission.” J Neurosci 18(5): 1904-12.
Liao G, W. J., Guo J, Allard J, Cheng J, Nguyen A, Shafer S, Puech A, McPherson J D, Foernzler D, Peltz G, Usuka J. (2004). “In silico genetics: Identification of a novel functional element regulating H2-E? Gene Expression.” Science in press.
Lindberg, P., L. Gunnarsson, et al. (1992). “Atelectasis and lung function in the postoperative period.” Acta Anaesthesiol Scand 36(6): 546-53.
Longnecker, D. E., P. A. Grazis, et al. (1973). “Naloxone for antagonism of morphine-induced respiratory depression.” Anesth Analg 52(3): 447-53.
MacDermott, A. B., L. W. Role, et al. (1999). “Presynaptic ionotropic receptors and the control of transmitter release.” Annu Rev Neurosci 22: 443-85.
Marubio, L. M., M. del Mar Arroyo-Jimenez, et al. (1999). “Reduced antinociception in mice lacking neuronal nicotinic receptor subunits.” Nature 398(6730): 805-10.
Miao, F. J., P. G. Green, et al. (2004). “Central terminals of nociceptors are targets for nicotine suppression of inflammation.” Neuroscience 123(3): 777-84.
Minto, C. F., T. W. Schnider, et al. (2000). “Response surface model for anesthetic drug interactions.” Anesthesiology 92(6): 1603-16.
Mitchell, S. N. (1993). “Role of the locus coeruleus in the noradrenergic response to a systemic administration of nicotine.” Neuropharmacology 32(10): 937-49.
Ng, A., J. Parker, et al. (2002). “Does the opioid-sparing effect of rectal diclofenac following total abdominal hysterectomy benefit the patient?” Br J Anaesth 88(5): 714-6.
O'Hara, D. A., G. Fanciullo, et al. (1997). “Evaluation of the safety and efficacy of ketorolac versus morphine by patient-controlled analgesia for postoperative pain.” Pharmacotherapy 17(5): 891-9.
Owen, H., V. McMillan, et al. (1990). “Postoperative pain therapy: a survey of patients' expectations and their experiences.” Pain 41(3): 303-7.
Papke, R. L., M. Bencherif, et al. (1996). “An evaluation of neuronal nicotinic acetylcholine receptor activation by quaternary nitrogen compounds indicates that choline is selective for the alpha 7 subtype.” Neurosci Lett 213(3): 201-4.
Parvini, S., S. R. Hamann, et al. (1993). “Pharmacologic characteristics of a medullary hyperalgesic center.” J Pharmacol Exp Ther 265(1): 286-93.
Perkins, K., J. Grobe, et al. (1994). “Effects of Nicotine on Thermal Pain Detection in Humans.” Experimental and Clinical Psychopharmacology 2(1): 95-106.
Pogatzki, E. M. and S. N. Raja (2003). “A mouse model of incisional pain.” Anesthesiology 99(4): 1023-7.
Pomerleau, O. F. (1986). “Nicotine as a psychoactive drug: anxiety and pain reduction.” Psychopharmacol Bull 22(3): 865-9.
Pomerleau, O. F., D. C. Turk, et al. (1984). “The effects of cigarette smoking on pain and anxiety.” Addict Behav 9(3): 265-71.
Rao, T. S., L. D. Correa, et al. (1996). “Evaluation of anti-nociceptive effects of neuronal nicotinic acetylcholine receptor (NAChR) ligands in the rat tail-flick assay.” Neuropharmacology 35(4): 393-405.
Roberts, R. G., J. E. Stevenson, et al. (1995). “Nicotinic acetylcholine receptors on capsaicin-sensitive nerves.” Neuroreport 6(11): 1578-82.
Roszkowski, M. T., J. Q. Swift, et al. (1997). “Effect of NSAID administration on tissue levels of immunoreactive prostaglandin E2, leukotriene B4, and (S)-flurbiprofen following extraction of impacted third molars.” Pain 73(3): 339-45.
Rueter, L. E., M. D. Meyer, et al. (2000). “Spinal mechanisms underlying A-85380-induced effects on acute thermal pain.” Brain Res 872(1-2): 93-101.
Saray, A., U. Buyukkocak, et al. (2001). “Diclofenac and metamizol in postoperative analgesia in plastic surgery.” Acta Chir Plast 43(3): 71-6.
Schmidt, B. L., C. H. Tambeli, et al. (2001). “Nicotine withdrawal hyperalgesia and opioid-mediated analgesia depend on nicotine receptors in nucleus accumbens.” Neuroscience 106(1): 129-36.
Schneider, N. G., E. Lunell, et al. (1996). “Clinical pharmacokinetics of nasal nicotine delivery. A review and comparison to other nicotine systems.” Clin Pharmacokinet 31(1): 65-80.
Sennholz, G. (2000). “Bispectral analysis technology and equipment.” Minerva Anestesiol 66(5): 386-8.
Short, T. G., T. Y. Ho, et al. (2002). “Efficient trial design for eliciting a pharmacokinetic-pharmacodynamic model-based response surface describing the interaction between two intravenous anesthetic drugs.” Anesthesiology 96(2): 400-8.
Shytle, R. D., T. Mori, et al. (2004). “Cholinergic modulation of microglial activation by alpha 7 nicotinic receptors.” J Neurochem 89(2): 337-43.
Stephens, J., B. Laskin, et al. (2003). “The burden of acute postoperative pain and the potential role of the COX-2-specific inhibitors.” Rheumatology (Oxford) 42 Suppl 3: iii40-52.
Sutherland, G., M. A. Russell, et al. (1992). “Nasal nicotine spray: a rapid nicotine delivery system.” Psychopharmacology (Berl) 108(4): 512-8.
Svensson, I., B. Sjostrom, et al. (2000). “Assessment of pain experiences after elective surgery.” J Pain Symptom Manage 20(3): 193-201.
Swift, J. Q., M. G. Garry, et al. (1993). “Effect of flurbiprofen on tissue levels of immunoreactive bradykinin and acute postoperative pain.” J Oral Maxillofac Surg 51(2): 112 -6; discussion 116-7.
Thomas, T., C. Robinson, et al. (1998). “Prediction and assessment of the severity of post-operative pain and of satisfaction with management.” Pain 75(2-3): 177-85.
Twycross, R. (1999). Opioids. Textbook of Pain. P. Wall and R. Melzack. Edinburgh, Churchill Livingstone: 1197.
van Haaren, F., K. G. Anderson, et al. (1999). “GTS-21, a mixed nicotinic receptor agonist/antagonist, does not affect the nicotine cue.” Pharmacol Biochem Behav 64(2): 439-44.
Waldhoer, M., S. E. Bartlett, et al. (2004). “Opioid receptors.” Annu Rev Biochem 73: 953-90.
Wang, H., M. Yu, et al. (2003). “Nicotinic acetylcholine receptor alpha7 subunit is an essential regulator of inflammation.” Nature 421(6921): 384-8.
Woodside, J. R. (2000). “Female smokers have increased postoperative narcotic requirements.” J Addict Dis 19(4): 1-10.
Zahn, P. K. and T. J. Brennan (1999). “Primary and secondary hyperalgesia in a rat model for human postoperative pain.” Anesthesiology 90(3): 863-72.
Zarrindast, M. R., M. R. Khoshayand, et al. (1999). “The development of cross-tolerance between morphine and nicotine in mice.” Eur Neuropsychopharmacol 9(3): 227-33.
Flood P and Daniel D. Nasal Nicotine for the Treatment of Postoperative Pain. (2004) Anesthesiology—in press. Rowley T J, Daniel D and Flood P. Role of Adrenergic and Cholinergic Transmission in Volatile Anesthetic Induced Pain Enhancement. (2004) Anesth Analg.—in press.
Damaj M I, Flood P, Ho K K, May E L, and Martin B R. Effect of Dextrometorphan and Dextrorphan on Nicotine and Neuronal Nicotinic Receptors: In Vitro and in Vivo Selectivity. (2004) J Pharmacol Exp Ther.—in press.

1. A method for reducing, or inhibiting the onset of, pain in a subject comprising administering to the subject
(a) a nicotinic receptor agonist, and (b) an opioid receptor agonist;
wherein the ratio of nicotinic receptor agonist to opioid receptor agonist administered to the subject is less than 3:4 and greater than 1:100, so as to thereby reduce, or inhibit the onset of, pain in the subject.

2. A method for reducing, or inhibiting the onset of, pain in a subject comprising administering to the subject
(a) a nicotinic receptor agonist at a rate of less than 3 mg per three hour period; and
(b) an opioid receptor agonist at a rate of less than 4 mg per three hour period,
so as to thereby reduce, or inhibit the onset of, pain in the subject.

3.-21. (canceled)

22. The method of claim 1, wherein the nicotinic receptor agonist is selected from the group consisting of nicotine, meta-nicotine, DMBX-anabaseine, anabaseine, choline, acetylcholine, epibatidine and cytisine.

23. The method of claim 22, wherein the nicotinic receptor agonist is nicotine.

24. The method of claim 1, wherein the opioid receptor agonist is selected from the group consisting of morphine, meperidine, fentanyl, hydromorphone, alfentanil, remifentanil, carfenanil, sufenanil, butorphanol, buprenorphine and pentazocine.

25. The method of claim 24, wherein the opioid receptor agonist is morphine.

26. The method of claim 1, wherein the nicotinic receptor agonist is nicotine and the opioid receptor agonist is morphine.

27.-44. (canceled)

45. A composition comprising a pharmaceutically acceptable carrier, a nicotinic receptor agonist and an opioid receptor agonist, wherein the nicotinic receptor agonist and the opioid receptor agonist are present in a ratio of between greater than 1:100 and less than 3:4.

46.-53. (canceled)
